(19) United States
(12) Patent Application Publication
(10) Pub. No.: US 2024/0400503 A1
(43) Pub. Date: Dec. 5, 2024

(54) NOVEL PHOTOINITIATORS

(71) Applicant: IGM RESINS ITALIA S.R.L, Milano (MI) (IT)

(72) Inventors: Marika MORONE, Lipomo (CO) (IT); Vincenzo RAZZANO, Bussero (MI) (IT); Stefan Jean Yves Marie VAN DEN BRANDEN, Milan (MI) (IT)

(21) Appl. No.: 18/698,759

(22) PCT Filed: Aug. 4, 2022
(86) PCT No.: PCT/EP2022/071953

§ 371 Date: Apr. 4, 2024

(30) Foreign Application Priority Data
Oct. 8, 2021 (IT) 102021000025868

(51) Int. Cl. C07C 319/20; C08F 2/50; C09D 11/101

(52) U.S. Cl. CPC C07C 319/20; C08F 2/50; C09D 11/101

(57) ABSTRACT

The present invention relates to novel photoinitiators having improved performances and to their use in photopolymerization compositions. The invention also relates to a process for the photopolymerization of compositions comprising said photoinitiators as well as their use in articles of manufacture, including printed, coated, and fabricated assemblies.

The present invention relates to novel photoinitiators having improved reactivity and surface curing and/or lower post-cure yellowing and to their use in photopolymerization compositions. The invention also concerns a process for the photopolymerization of compositions comprising said photoinitiators as well as their use in articles of manufacture, including printed, coated, and fabricated assemblies.

### PRIOR ART

In the last years, the design and the development of new photoinitiators (PIs) has been attracted increasing attention due to the large amount of photoinitiators that have been or are going to be banned as toxic or reprotoxic.

Various attempts were made to develop new photoinitiators able to mimic standard photoinitiators or to overcome problems such as yellowing, higher line speed, curing under LED lamps, some examples are glyoxylate 3-ketocoumarins (WO2021070152), Benzoyl phenyltelluride PIs (Macromolecules, 2014, 47(16), 5526-5531), silicon based PIs (JP2010229169, Macromolecules, 2009, 42(16), 6031-6037, Macromolecules 2007, 40(24), 8527-8530, Macromol. Rapid Commun. 2017, 38, 1600470, Macromolecules, 2017, 50(17), 6911-6923), fluorine based PIs (US2019/0155153).

Unfortunately, when used in standard applications, also these photoinitiators show some limitations, such as low surface curing and, most importantly, post-cure yellowing, which makes said PI inappropriate in polymerizable systems involving transparent varnishes.

So, there exists the need of new technical solutions able to improve the surface curing of PIs without affecting the good reactivity of these products and/or to limit post-cure yellowing.

### AIMS OF THE INVENTION

It is a first aim of the invention to provide for novel PIs, their use as photoinitiators and photocurable compositions comprising them.

It is a further aim of the invention to provide for photocurable compositions comprising the novel PIs of the invention.

Is a further aim of the invention to provide for processes for photocuring ethylenically unsaturated compounds using the novel PIs of the invention, as well as articles of manufacture made by said process.

### DESCRIPTION OF THE INVENTION

Surprisingly, we found that certain difunctional photoinitiators well react to UVA, UVB and UVC wavelength ranges as well as, and more preferably react to LED sources emitting in the range from 350 to 420 nm, maintaining a low post cure yellowing representing a technical progress compared to prior art.

In fact, the increased performance under LED lamp is always associated to an high post-cure yellowing.

According to one of its aspects, the present invention relates to a compound of formula (I)

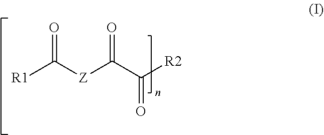

wherein
R1 is selected from aryl and heteroaryl, each being optionally substituted by one or more substituents selected from:
halogen, substituted or unsubstituted C1-C20 alkyl, substituted or unsubstituted C1-C20 alkoxy, substituted or unsubstituted C1-C20 alkylthio, substituted or unsubstituted (C1-C12alkyl)₂amino, morpholino, piperidino, piperazino and N(C1-C12alkyl)piperazino;
n is from 2 to 8;
R2 is a group of formula (II)

*-G-[OH]p (II)
wherein
G is the residue of optionally ethoxylated and/or propoxylated monomeric, oligomeric or polymeric polyols G-(OH)ₙ₊ₚ;
p is from 0 to 7;
n+p is from 2 to 8;
the star shows the linking bond to the compound of formula (I);
Z is selected from (i), (ii) and (iii)

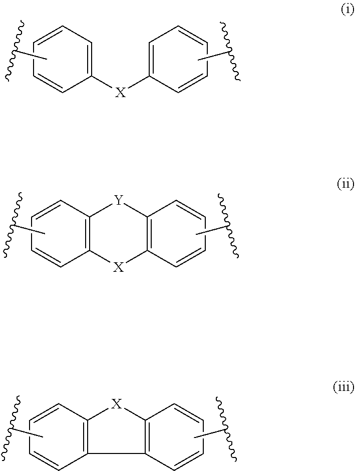

wherein
X is selected from O, S and C(R3)(R4);
Y is selected from O, S, C(R3)(R4) and NR5;
R3 and R4 each independently, are selected from H, C1-C12 alkyl, OH and C1-C10 alkoxy;
R5 is selected from H and C1-C8 alkyl;
and the wavy line shows the bonds linking to the keto groups of formula (I); provided that when Z is (iii) and X is C(R4)(R5), then R1 is a heteroaryl selected from an optionally substituted coumarin or a benzocoumarin.

According to the invention, G-(OH)ₙ₊ₚ is selected from a monomeric, an oligomeric, a polymeric polyol and mixtures thereof, which can be optionally ethoxylated or propoxylated.

Examples of suitable monomeric and oligomeric polyols are ethylene glycol, propylene glycol, 1,2-butandiol, 1,2-propandiol, 1,2-hexandiol, diethanolamine, N-methyldiethanolamine, glycerol, di-glycerol, tri-glycerol, triethanolamine, trimethylol propane, di-trimethylol propane, pentaerythritol, di-pentaeritrithol, sugar alcohols, such as sorbitol, mannitol and xylitol, mixtures thereof.

Examples of polymeric polyols are polyhydroxy polyethers, which can be both aliphatic or aromatic, polyhydroxy polyesters, polyhydroxy polyamides, polyhydroxy polyimides, polyhydroxy polycarbonates, styrene-allyl alcohols copolymers.

The alkoxylated polyols are particularly preferred for the realization of the present invention. Examples of such alkoxylated polyols are monomeric, oligomeric and polymeric polyols mentioned above, which have been alkoxylated, for example ethoxylated and/or propoxylated and/or butoxylated. Other suitable examples are linear or branched polyamines, which have been alkoxylated, and polyalkoxylated diamines, such as ethoxylated ethylene diamine and ethoxylated 1,3-propylene diamine. In the alkoxylated compounds of the invention, each group reactive toward the alkylene oxide can bring from 0 to 15 alkoxy units, preferably from 1 to 6 alkoxy units.

In a preferred embodiment G-(OH)ₙ₊ₚ is selected from monomeric and oligomeric polyols.

In another preferred embodiment G-(OH)ₙ₊ₚ is selected from monomeric and oligomeric polyols which have been ethoxylated and/or propoxylated.

Preferably, G-(OH)ₙ₊ₚ has a number average molecular weight
not greater than 1,500 Da, more preferably not greater than 1,000 Da and most preferably not greater than 800 Da; and
not lower than 100 Da, preferably not lower than 200 Da.

Preferably, G-(OH)ₙ₊ₚ is selected from glycerol, ethoxylated and/or propoxylated glycerol, di-glycerol, ethoxylated and/or propoxylated di-glycerol, trimethylolpropane, ethoxylated and/or propoxylated trimethylolpropane, di-trimethylolpropane, ethoxylated and/or propoxylated di-trimethylolpropane, penthaerythritol, ethoxylated and/or propoxylated penthaerythritol, di-penthaerythritol, ethoxylated and/or propoxylated di-penthaerythritol, sorbitol, ethoxylated and/or propoxylated sorbitol, triethanolamine, and ethoxylated and/or propoxylated triethanolamine.

Preferably, n+p is from 2 to 8, and more preferably from 3 to 6, for instance, 3, 4, 5 or 6.

Preferably, n is from 2 to 6 and more preferably from 2 to 4, for instance, 2, 3 or 4.

Preferably, p is from 0 to 6 and more preferably from 0 to 3, for instance, 0, 1, 2, or 3.

When p is different from 0, compounds of Formula (I) have free alcoholic groups.

When R1 is an aryl or a heteroaryl substituted by more than one substituent, then said substituents may be the same or different. Preferably, R1 is aryl, more preferably phenyl non-substituted or substituted by C1-C20 alkyl.

Preferably, R1 is selected from phenyl, trimethylphenyl, halophenyl preferably fluorophenyl, biphenyl, coumarin-3-yl, naphthyl and thiophenyl.

According to an embodiment, R1 is a non-substituted coumarin or a benzocoumarin.

Mixtures of compounds of formula (I) are also included in the scope of the invention, for instance, but not limited to, when R2 is a group of formula (II), mixtures of compounds of formula (I) wherein p is zero and compounds of formula (I) wherein p is other than zero.

According to preferred embodiment, Z is (i) and X is S or O, preferably S. When X is S, oxidized forms of sulfur to sulfone or sulfoxide are also encompassed within the scope of the protection of the present invention.

According to a preferred embodiment of the present invention:
n is from 2 to 6, more preferably from 2 to 4, for instance, 2, 3 or 4;
R2 is a group of formula (II);
p is from 0 to 6, more preferably from 0 to 3, for instance 0, 1, 2 or 3;
n+p is from 2 to 8 more preferably from 3 to 6, for instance 3, 4, 5 or 6.

According to a preferred Embodiment (1), X is S or O, more preferably S. According to a preferred Embodiment (1), R1 is aryl, more preferably phenyl non-substituted or substituted by C1-C20 alkyl.

According to an embodiment, herein referred to as Embodiment (1), Z is (i), R2 is a group of formula (II) and the other variables are as above defined.

According to a preferred Embodiment (1), X is S or O, more preferably S.

According to a preferred Embodiment (1), G-(OH)ₙ₊ₚ is a monomeric or oligomeric polyol which has been ethoxylated and/or propoxylated.

According to an alternative preferred Embodiment (1), n is from 2 to 5, more preferably from 2 to 4, for instance 2, 3 or 4; R2 is a group of formula (II); p is from 0 to 3, more preferably from 0 to 2, for instance 0, 1 or 2; and n+p is from 2 to 6, more preferably from 3 to 5, for instance 3, 4 or 5.

According to a preferred Embodiment (1), R1 is aryl, more preferably phenyl non-substituted or substituted by C1-C20 alkyl.

According to a preferred Embodiment (2), Z is (ii), X is O and Y is C(R4)(R5) and R4 and R5 are C1-C12alkyl, R1 is aryl, more preferably phenyl non-substituted or substituted by C1-C20 alkyl and G-(OH)ₙ₊ₚ is a monomeric or oligomeric polyol which has been ethoxylated and/or propoxylated

According to an alternative preferred Embodiment (2), n is from 2 to 5, more preferably from 2 to 4, for instance 2, 3 or 4; R2 is a group of formula (II); p is from 0 to 3, more preferably from 0 to 2, for instance 0, 1 or 2; and n+p is from 2 to 6, more preferably from 3 to 5, for instance 3, 4 or 5.

According to an embodiment, herein referred to as Embodiment (3), Z is (iii), R2 is a group of formula (II) and the other variables are as above defined.

According to a more preferred Embodiment (3), X is O or S.

According to a preferred Embodiment (3), G-(OH)ₙ₊ₚ is a monomeric or oligomeric polyol which has been ethoxylated and/or propoxylated.

According to an alternative preferred Embodiment (3), n is from 2 to 5, more preferably from 2 to 4, for instance 2, 3 or 4; R2 is a group of formula (II); p is from 0 to 3, more preferably from 0 to 2, for instance 0, 1 or 2; and n+p is from 2 to 6, more preferably from 3 to 5, for instance 3, 4 or 5.

According to a preferred Embodiment (3), R1 is aryl, more preferably phenyl non-substituted or substituted by C1-C20 alkyl.

Where possible, within each Embodiment (1) to (3), all the preferred embodiments may be combined.

In the present description the expressions “alkyl” or “alkyl group” mean, where not differently indicated, a linear or branched, saturated alkyl chain containing the given number of carbon atoms and includes all possible isomer for each number of carbon atoms in the alkyl group, i.e. for three carbon atoms: n-propyl and isopropyl; for four carbon atoms: n-butyl, isobutyl and tertiary-butyl; for five carbon atoms: n-pentyl, 1,1-dimethyl-propyl, 2,2-dimethylpropyl and 2-methyl-butyl, etc.

“Alkenyl” or “alkenyl group” mean an unsaturated group containing from 2 to 12 carbon atoms, preferably C3 to C12 carbon atoms, which can be, for example, allyl, methallyl or undecenyl.

The expressions “cycloalkyl” or “cycloalkyl group” mean, where not differently indicated, an aliphatic ring preferably containing 5 or 6 carbon atoms which can be cyclopentyl or cyclohexyl.

The expressions “aryl” or “aryl group” include, but are not limited to, a substituted or unsubstituted phenyl group, a substituted or unsubstituted naphthyl group, an anthracenyl group, an indenyl group, a fluorenyl group, a gem-dialkyl fluorenyl group, a phenanthracenyl group, preferably a substituted or unsubstituted phenyl group, a substituted or unsubstituted naphthyl group or a substituted or unsubstituted phenanthracenyl group, more preferably a phenyl group.

The expressions “heteroaryl” or “heteroaryl group” include, but are not limited to, furan, thiophene, pyrrole, N-substituted pyrrole, oxazole, isooxazole, thiazole, isothiazole, imidazole, pyrazole, pyran, pyridine, pyrrolidine, indole, N-substituted indole, dibenzofuran, benzocarbazole, quinoline, isoquinoline, coumarin and others. Preferably heteroaryl are substituted or unsubstituted furan, thiophene, pyrrole coumarin and a benzocoumarin, each of them being unsubstituted or substituted, preferably substituted or unsubstituted coumarin.

Coumarin has the following formula:

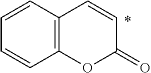 [SMILES: O=c1ccc2ccccc2o1]

and it is bound to the rest of the molecule in position 3, as indicated by the star.

Benzocoumarins have the following formulas:

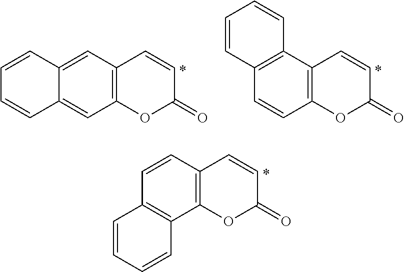

and they are bound to the rest of the molecule in position 3, as indicated by the star.

When a group is defined as substituted, if not otherwise specifically defined, the term “substituted” herein means that said group bears one or more substituents, said substituents being preferably selected from halogen atoms, alkyl, aryl, cycloalkyl, alkoxy, aryloxy, alkylamino, dialkylamino, alkylthio or arylthio, heterocyclic and phosphorous or silicon containing groups. More preferably the term “substituted” herein indicates one or more groups selected from methyl, ethyl, isopropyl, tert-butyl, phenyl, trifluoromethyl, cyano, acetyl, ethoxycarbonyl, carboxyl, carboxylate, amino, methylamino, dimethylamino, ethylamino, diethylamino, isopropylamino, diisopropylamino, cyclohexylamino, dicyclohexylamino, acetylamino, piperidino, pyrrolidyl, methoxy, ethoxy, propoxy, isopropoxy, butoxy, pentyloxy, phenoxy, hydroxyl, acetoxy, —PO₃H, methylthio, ethylthio, i-propylthio, n-propylthio, phenyltio, mercapto, acetylthio, thiocyano, methylsulfinyl, methylsulfonyl, dimethylsulfonyl, sulfonate groups, fluorine atom, chlorine atom, bromine atom, iodine atom, trimethylsilyl, pentamethyldisilyl, triethylsilyl, trimethylstannyl, furyl, thienyl, pyridyl and morpholino.

The compounds of the invention can be prepared according to any suitable process. For example, they can be prepared by double Friedel-Crafts acylation and optionally a transesterification according to Scheme 1 below:

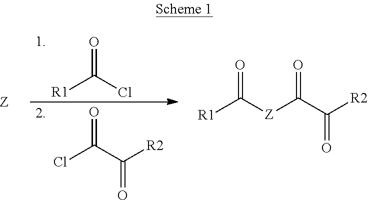

According to another of its aspects, the present invention relates to a process for the preparation of compounds of formula (I) which comprises performing Friedel Crafts acylations on the Z groups (i), (ii) or (iii), and optionally a transesterification according to Scheme 1 above.

The skilled in the art is perfectly able to perform the chemical reactions of Scheme 1, according to the known methods.

Details of the process of the invention are reported in the Experimental Section of the present description.

Preferred PIs according to the invention are the following:

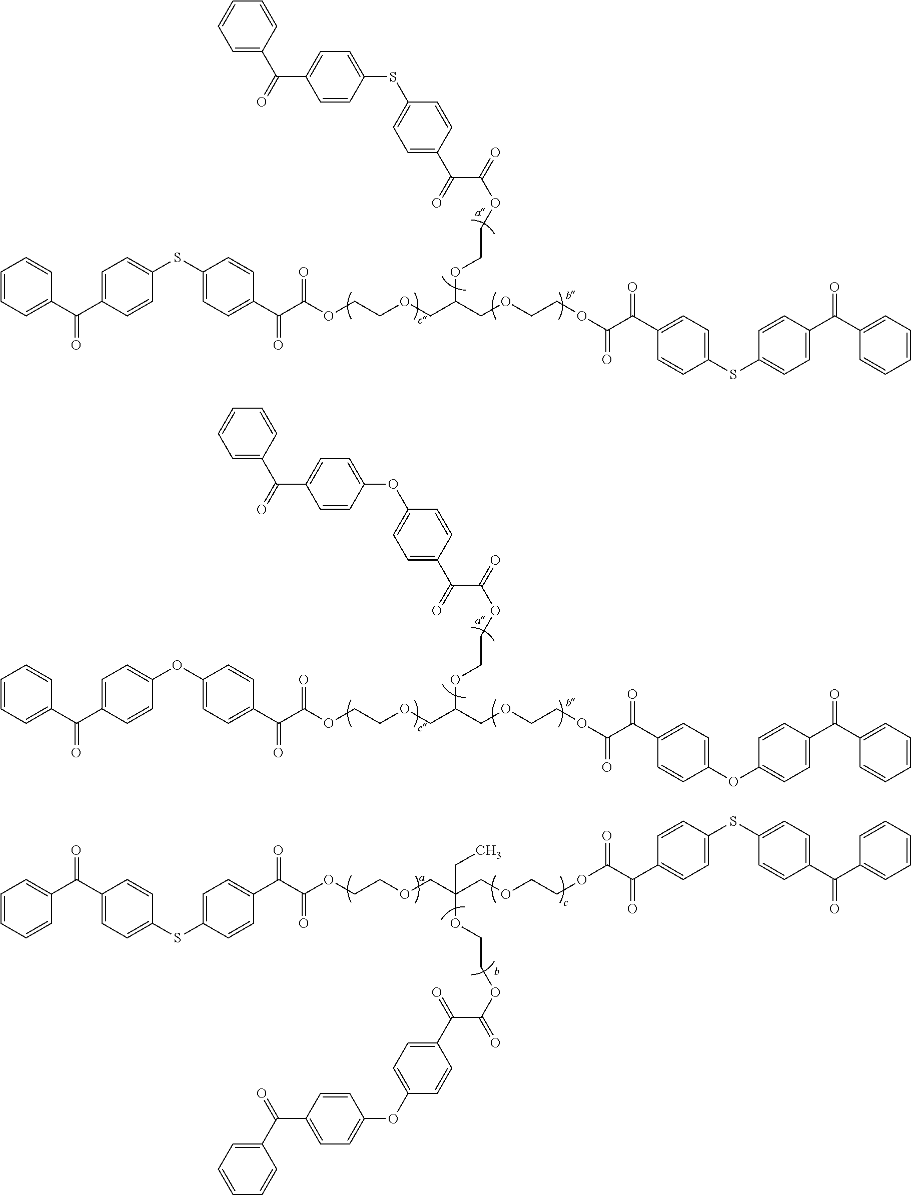

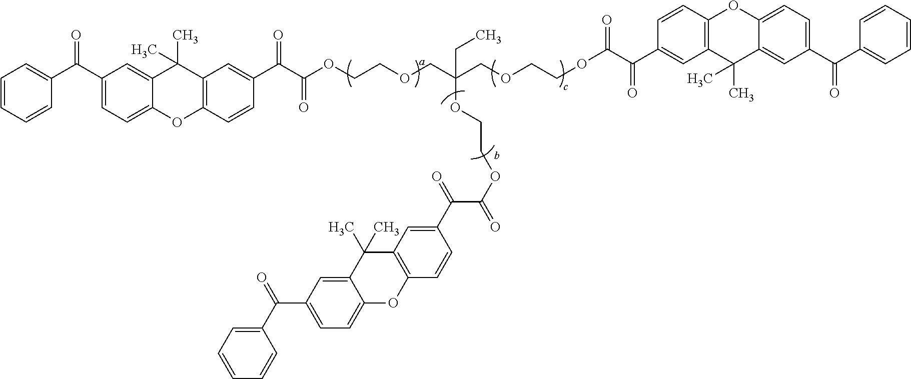

In all this specification, letters (a), (a′), (a″) and subsequent letters represent the number of ethoxy units and are, each independently, from 0 to 15 more preferably from 1 to 8.

According to another of its aspects, the present invention relates to a photopolymerizable composition comprising:
a) from 50 to 99.9%, preferably from 70 to 98.9% by weight, based on the total content of the composition, of at least one ethylenically unsaturated compound;
b) from 0.1 to 35%, preferably from 0.1 to 20%, and more preferably from 0.2 to 15% by weight, based on the total content of the composition, of at least one compound of formula (I) as above defined; and
c) from 0 to 20% by weight, preferably from 0 to 15%, and more preferably from 0.2 to 15% by weight, based on the total content of the composition, of an accelerator and/or of a coinitiator.

According to the present invention, the terms “photocuring” and “photopolymerizing” and related terms, are synonyms.

The expression “based on the total content of the composition” means that the % weight amounts of any of the components is calculated with respect to the sum of the weight of all the components of the composition, including any possible further additional components (in addition to a), b) and c) above), but possible water and/or solvents which may be present in the composition are not considered for the calculation of said % weight amounts.

According to another of its aspects, the present invention relates to a process for photocuring photopolymerizable compositions coatings, adhesives and inks, which process comprises:
i. providing a photopolymerizable composition as above defined;
ii. coating or printing said photopolymerizable composition onto a substrate, and
iii. photocuring said coated or printed composition with a light source on said substrate.

According to another of its aspects, the present invention relates to a process for three-dimensional printing which comprising photocuring with a light source a mixture comprising the composition as above defined.

According to another of its aspects, the present invention relates to an article of manufacture obtained by the process of the invention.

The photocurable compositions of the present invention can also comprise one or more of the following components: (d) photosensitizers and/or (e) further photoinitiators and/or (f) conventional additives, in addition to compounds (a), (b) and, when present, (c).

According to a preferred embodiment, the photopolymerizable composition used the processes of the invention comprises at least components (a), (b), (c), more preferably at least (a), (b), (c), and (d) as above defined.

The photoinitiators of the invention can be used in photocurable compositions comprising ethylenically unsaturated compounds (a). Said unsaturated compounds (a) can contain one or more olefinic double bonds. They can be low-molecular weight (monomeric) or high-molecular weight (oligomeric) compounds.

Examples of suitable low molecular weight monomers (monomeric compounds) having one double bond are alkyl or hydroxyalkyl acrylates or methacrylates, such as methyl-, ethyl-, butyl-, 2-ethylhexyl-,2-hydroxyethyl- or isobornyl-acrylate; and methyl or ethyl methacrylate. Further examples are resins modified with silicon or fluorine, e.g. silicone acrylates. Further examples of these monomers are acrylonitrile, acrylamide, methacrylamide, N-substituted (meth)acrylamides, styrene, alkylstyrenes and halogeno styrenes, vinyl esters such as vinyl acetate, vinyl ethers such as iso-butyl vinyl ether, N-vinylpyrrolidone, vinyl chloride or vinylidene chloride.

Examples of monomers having more than one double bond are the ethylene glycol diacrylate, propylene glycol diacrylate, neopentyl glycol diacrylate, hexamethylene glycol diacrylate, bisphenol A diacrylate, 4,4′-bis-(2-acryloyloxyethoxy)-diphenylpropane, trimethylolpropane triacrylate, pentaerythritol triacrylate or tetraacrylate, vinyl acrylate, divinyl benzene, divinyl succinate, diallyl phthalate, triallyl phosphate, triallyl isocyanurate or tris-(2-acryloylethyl) isocyanurate.

Examples of high-molecular weight (oligomeric) polyunsaturated compounds are acrylated epoxy resins, acrylated or vinyl-ether- or epoxy-group-containing polyesters, acrylated polyurethanes or acrylated polyethers.

Further examples of unsaturated oligomers are unsaturated polyester resins which are usually prepared from maleic acid, phthalic acid and one or more diols and which have molecular weights of from about 500 Da to 3,000 Da. Such unsaturated oligomers can also be referred to as prepolymers.

Examples of compounds (a) which are particularly suitable for the implementation of the present invention, are esters of ethylenically unsaturated carboxylic acids and polyols or polyepoxides, and polymers containing ethylenically unsaturated groups in the chain or in side groups, e.g. unsaturated polyesters, polyamides and polyurethanes and copolymers thereof, alkyl resins, polybutadiene and butadiene copolymers, polyisoprene and isoprene copolymers, polymers and copolymers having (meth)acrylic groups in side chains, as well as mixtures thereof.

Illustrative examples of unsaturated carboxylic acids or anhydrides, useful for the preparation of the above esters, are acrylic acid, methacrylic acid, maleic anhydride, crotonic acid, itaconic acid, cinnamic acid and unsaturated fatty acids such as linolenic acid and oleic acid. Acrylic and methacrylic acid are preferred.

Examples of polyols, which can also be esterified, are aromatic and aliphatic and cycloaliphatic polyols, preferably aliphatic and cycloaliphatic polyols.

Aromatic polyols are, for example, hydroquinone, 4,4′-dihydroxydiphenyl, 2,2-di(4-hydroxyphenyl) propane, as well as novolaks and resoles.

Polyepoxides, which can be esterified, include those based on the said polyols, especially the reaction products between aromatic polyols and epichlorohydrin. Also suitable as polyols are polymers and copolymers that contain hydroxyl groups in the polymer chain or inside groups, for example polyvinyl alcohol and copolymers thereof or polymethacrylic acid hydroxyalkyl esters or copolymers thereof. Further suitable polyols are oligoesters carrying hydroxyl terminal groups.

Examples of aliphatic and cycloaliphatic polyols include alkylenediols containing preferably from 2 to 12 carbon atoms, such as ethylene glycol, 1,2- or 1,3-propanediol, 1,2-, 1,3- or 1,4-butanediol, pentanediol, hexanediol, octanediol, dodecanediol, diethylene glycol, triethylene glycol, polyethylene glycols having molecular weights of preferably from 200 Da to 1,500 Da, 1,3-cyclopentanediol, 1,2-, 1,3- or 1,4-cyclohexanediol, 1,4-dihydroxymethyl cyclohexane, glycerol, tris(p-hydroxy-ethyl)amine, trimethylolethane, trimethylolpropane, pentaerythritol, dipentaerythritol and sorbitol.

Further suitable ethylenically unsaturated compounds (a) are unsaturated polyamides obtained from unsaturated carboxylic acids and aromatic, aliphatic and cycloaliphatic polyamines having preferably from 2 to 6, preferably from 2 to 4, amino groups. Examples of such polyamines are: ethylenediamine, 1,2- or 1,3-propylenediamine, 1,2-, 1,3- or 1,4-butylenediamine, 1,5-pentylenediamine, 1,6-hexylenediamine, octylenediamine, dodecylene diamine, 1,4-diaminocyclohexane, isophoronediamine, phenylene diamine, bisphenylenediamine, di-(β-aminoethyl) ether, diethylene triamine, triethylenetetramine and di(β-aminoethoxy)- and di(β-aminopropoxy)ethane. Other suitable polyamines are polymers and copolymers which may contain additional amino groups in the side chain and oligoamides containing amino end groups.

Specific examples of such unsaturated polyamides are methylenebisacrylamide, 1,6-hexamethylene bisacrylamide, diethylenetriamine trismethacrylamide, bis(methacrylamidopropoxy) ethane and N—[(β-hydroxyethoxy)ethyl]-acrylamide.

Unsaturated polyurethanes are also suitable for the implementation of the present invention as components (a), for example those derived from saturated or unsaturated diisocyanates and unsaturated or saturated diols. Polybutadiene and polyisoprene and copolymers thereof may also be used. Suitable monomers include, for example, olefins, such as ethylene, propene, butene and hexene, (meth)acrylates, acrylonitrile, styrene and vinyl chloride. Polymers having unsaturated (meth)acrylate groups in the side chain can also be used as component (a). They may typically be reaction products of epoxy resins based on novolac with (meth)acrylic acid; homo- or copolymers of vinyl alcohol or hydroxyalkyl derivatives thereof that have been esterified with (meth)acrylic acid; and homo- and co-polymers of (meth)acrylates that have been esterified with hydroxyalkyl (meth)acrylates.

According to a preferred embodiment, the photocurable composition further comprises a coinitiators (c), also referred to as accelerators.

Suitable examples of accelerators/coinitiators (c) are alcohols, thiols, thioethers, amines or ethers that have an available hydrogen, bonded to a carbon adjacent to the heteroatom, disulfides and phosphines, e.g. as described in EP 438 123 and GB 2 180 358.

Suitable examples of amine accelerators/co-initiators include, but are not limited to, aliphatic, cycloaliphatic, aromatic, aryl-aliphatic, heterocyclic, oligomeric or polymeric amines. They can be primary, secondary or tertiary amines, for example butyl amine, dibutyl amine, tributyl amine, cyclohexyl amine, benzyldimethyl amine, di-cyclohexyl amine, N-phenyl glycine, triethyl amine, phenyl-diethanol amine, triethanolamine, piperidine, piperazine, morpholine, pyridine, quinoline, esters of dimethylamino benzoic acid, Michler's ketone (4,4′-bis-dimethyl aminobenzophenone) and derivatives thereof.

Preferably said accelerator and/or coinitiator is an amine, preferably a tertiary amine.

As the amine accelerators/co-initiators, an amine-modified acrylate compound can be used; examples of such amine-modified acrylate include acrylates modified by reaction with a primary or secondary amine that are described in U.S. Pat. No. 3,844,916, EP 280222, U.S. Pat. No. 5,482,649 or U.S. Pat. No. 5,734,002, Multifunctional amine and polymeric amine derivatives are also suitable as co-initiators some examples are Omnipol® ASA from IGM Resins B.V., Genopol® AB-2 from Rahn A.G., Speedcure® 7040 from Lambson Limited or those described in US2013/0012611.

The photocurable compositions of the present invention can also be formulated in compositions further comprising water and/or solvents, such as organic solvents.

Photosensitizers (d) can be present in an amount comprised between 0.01 and 15% by weight, based on the total content of the composition, preferably between 0.01 and 10% by weight.

Examples of photosensitizers are those commonly used in the art, aromatic carbonyl compounds, e.g. benzophenones, thioxanthones, anthraquinones, coumarins and 3-acylcoumarin derivatives, terphenyls, styryl ketones, and 3-(aroylmethylene)-thiazolines, camphorquinones and also eosin, rhodamine and erythrosine dyes.

Examples of thioxanthones are thioxanthone, 2-isopropyl thioxanthone, 2-chloro thioxanthone, 2-dodecyl thioxanthone, 2,4-diethyl thioxanthone, 2,4-dimethyl thioxanthone, 1-methoxycarbonyl thioxanthone, 2-ethoxycarbonyl thioxanthone, 3-(2-methoxyethoxycarbonyl) thioxanthone, 4-butoxycarbonyl thioxanthone, 3-butoxycarbonyl-7-methyl thioxanthone, 1-cyano-3-chloro thioxanthone, 1-ethoxycarbonyl-3-chloro thioxanthone, 1-ethoxycarbonyl-3-ethoxy thioxanthone, 1-ethoxycarbonyl-3-amino thioxanthone, 1-ethoxycarbonyl-3-phenylsulfuryl thioxanthone, 3,4-di[2-(2-methoxyethoxy)ethoxycarbonyl] thioxanthone, 1-ethoxycarbonyl-3-(1-methyl-1-morpholinoethyl) thioxanthone, 2-methyl-6-dimethoxymethyl thioxanthone, 2-methyl-6-(1,1-dimethoxybenzyl) thioxanthone, 2-morpholinomethyl thioxanthone, 2-methyl-6-morpholinomethyl thioxanthone, N-allylthioxanthone-3,4-dicarboximide, N-octylthioxanthone-3,4-dicarboximide, N-(1,1,3,3-tetramethylbutyl)-thioxanthone-3,4-dicarboximide, 1-phenoxy thioxanthone, 6-ethoxycarbonyl-1-2-methoxythioxanthone, 6-ethoxycarbonyl-2-methylthioxanthone, thioxanthone-2-polyethylene glycol ester, 2-hydroxy-3-(3,4-dimethyl-9-oxo-9H-thioxanthon-2-yloxy)-N,N,N-trimethyl-1-propanaminium chloride, or those described in the patent application PCT/EP2011/069514, such as n-dodecyl-7-methyl-thioxanthone-3-carboxylate and N,N-disobutyl-7-methyl-thioxanthone-3-carbamide. Also suitable are polymeric thioxanthone derivatives (e.g. Omnipol® TX from IGM Resins B.V., Genopol® TX-1 from Rahn A.G., Speedcure® 7010 from Lambson Limited).

Example of benzophenones are benzophenone, 4-phenyl benzophenone, 4-methoxy benzophenone, 4,4′-dimethoxybenzophenone, 4,4′-dimethyl benzophenone, 4,4′-dichloro benzophenone, 4,4′-dimethylamino benzophenone, 4,4′-diethylamino benzophenone, 4-methyl benzophenone, 2,4,6-trimethyl benzophenone, 4-(4-methylthiophenyl) benzophenone, 3,3′-dimethyl-4-methoxy benzophenone, methyl 2-benzoyl benzoate, 4-(2-hydroxyethylthio) benzophenone, 4-(4-tolylthio) benzophenone, 4-benzoyl-N,N,N-trimethylbenzene methanaminium chloride, 2-hydroxy-3-(4-benzoylphenoxy)-N,N,N-trimethyl-1-propanaminium chloride monohydrate, 4-(13-acryloyl-1,4,7,10,13-pentaoxatridecyl) benzophenone, 4-benzoyl-N,N-dimethyl-N-[2-(1-oxo-2-propenyl)oxylethyl-benzene methanaminium chloride, or those described in U.S. Pat. No. 9,938,231 (e.g. Omnirad® 991 from IGM Resins B.V.).

Also suitable are polymeric benzophenone derivatives (e.g. Omnipol® BP, Omnipol® 2702 and Omnipol® 682 all from IGM Resins B.V., Genopole BP-2 from Rahn A.G. and Speedcure® 7005 from Lambson Limited).

Examples of 3-acylcoumarin derivatives are 3-benzoyl coumarin, 3-benzoyl-7-methoxy coumarin, 3-benzoyl-5,7-di(propoxy) coumarin, 3-benzoyl-6,8-dichloro coumarin, 3-benzoyl-6-chloro coumarin, 3,3′-carbonyl-bis[5,7-di(propoxy) coumarin], 3,3′-carbonyl-bis(7-methoxy coumarin), 3,3′-carbonyl-bis(7-diethylamino coumarin), 3-isobutyroyl coumarin, 3-benzoyl-5,7-dimethoxy coumarin, 3-benzoyl-5,7-diethoxy coumarin, 3-benzoyl-5,7-dibutoxy coumarin, 3-benzoyl-5,7-di(methoxyethoxy) coumarin, 3-benzoyl-5,7-di(allyloxy) coumarin, 3-benzoyl-7-dimethylamino coumarin, 3-benzoyl-7-diethylamino coumarin, 3-isobutyroyl-1,7-dimethylamino coumarin, 5,7-dimethoxy-3-(1-benzoyl) coumarin, 5,7-dimethoxy-3(1-benzoyl)-coumarin, 3-benzoylbenzo [f]coumarin, 7-diethylamino-3-thienoyl coumarin, 3-(4-cyanobenzoyl)-5,7-dimethoxy coumarin, or those described in EP2909243 and WO2017216699.

Examples of 3-(aroylmethylene) thiazolines are 3-methy-1,2-benzoylmethylene-β-benzo thiazoline, 3-methyl-2-benzoylmethylene-benzo thiazoline, 3-ethyl-2-propionylmethylene-β-benzo thiazoline.

Examples of other aromatic carbonyl compounds are acetophenone, 3-methoxyacetophenone, 4-phenylacetophenone, benzil, such as that described in WO 2013/164394, 2-acetylnaphthalene, 2-naphthaldehyde, 9,10-anthraquinone, 9-fluorenone, dibenzosuberone, xanthone, 2,5-bis(4-diethylaminobenzylidene) cyclopentanone, α-(para-dimethylamino benzylidene), ketones, such as 2-(4-dimethylamino-benzylidene)-indan-1-one or 3-(4-dimethylaminophenyl)-1-indan-5-yl-propenone, 3-phenylthiophthalimide, N-methyl-3,5-di(ethylthio) phthalimide.

Particularly preferred are thioxanthones, coumarins and 3-acylcoumarins.

It was observed that the above components (d) increase the activity of photoinitiators (b) without shortening the shelf life of the compositions.

Moreover, such compositions have the special advantage that an appropriate choice of the photosensitizer (d) allows the spectral sensitivity of photoinitioator (b) to be shifted to any desired wavelength region. The skilled in the art is able to select the suitable photosensitizer (d) to make the photoinitiator(s) (b) work at any desired wavelength region.

The further possible photoinitiators (e) can be present in an amount comprised between 0.5 and 15% by weight, of the total content of the composition, preferably between 1 and 10% by weight of the composition.

Examples of other suitable photoinitiators (e) are camphorquinone, benzophenone, benzophenone derivatives, acetophenone, acetophenone derivatives, dialkoxyacetophenones, α-hydroxyketones, α-aminoketones, 4-aroyl-1,3-dioxolanes, benzoin alkyl ethers and benzil ketals, e.g. benzil dimethyl ketal, ketosulfones, e.g 1-[4-[(4-benzoyl-phenyl)-thio]-phenyl]-2-methyl-2-[(4-methyl-phenyl)-sulfonyl]-propan-1-one (Esacure® 1001, from IGM Resins B.V.), 3-ketocoumarins, for example as described in EP2909243 and WO2017216699, phenylglyoxylates and derivatives thereof, dimeric phenyl glyoxylates, peresters, e.g. benzophenonetetracarboxylic acid peresters, for example as described in EP 126 541, acylphosphine photoinitiators (which can be selected from mono-acylphosphine oxides, bis-acylphosphine oxides, tris-acylphosphine oxides and multifunctional mono- or bisacylphosphine oxides), halomethyltriazines, hexaaryl bisimidazole/coinitiator systems, e.g. ortho-chlorohexaphenylbisimidazole in combination with 2-mercaptobenzothiazole, ferrocenium compounds or titanocenes, for example dicyclopentadienyl-bis(2,6-difluoro-3-pyrrolo-phenyl)titanium, O-acyloxime ester photoinitiators.

Examples of α-hydroxyketones and α-aminoketones are 1-hydroxy cyclohexylphenyl ketone, 2-hydroxy-2-methyl-1-phenyl-propane-1-one, 1-[4-(2-hydroxyethoxy)phenyl]-2-hydroxy-2-methyl-1-propane-1-one, 2-hydroxy-1-{4-[4-(2-hydroxy-2-methyl-propionyl)-benzyl]phenyl}-2-methyl-propane-1-one), 2-hydroxy-1-{4-[4-(2-hydroxy-2-methyl-propionyl)-phenoxy]-phenyl}-2-methyl-propan-1-one, 2-methyl-1-(4-methylthiophenyl)-2-morpholinopropane-1-one), 2-benzyl-2-dimethylamino-1-(4-morpholinophenyl)-butane-1-one, and (2-(dimethylamino)-2-[(4-methylphenyl)methyl]-1-[4-(4-morpholinyl) phenyl]-1-butanone).

Examples of O-acyloxime ester photoinitiators are 1,2-octanedione,1-[4-(phenylthio)phenyl]-2-(O-benzoyloxime), ethanone 1-[9-ethyl-6-(2-methylbenzoyl)-9H-carbazole-3-yl] 1-(O-acetyloxime) or those described in GB 2339571.

Examples of acylphosphine photoinitiators include, but are not limited to, bis(2,4,6-trimethylbenzoyl)phenylphosphine oxide, bis(2,6-dimethoxybenzoyl)-2,4,4-trimethylpentyl phosphine oxide, bis(2,4,6-trimethylbenzoyl)-(2,4-dipentyloxyphenyl), 2,4,6-trimethylbenzoyl-diphenyl phosphine oxide and ethyl (2,4,6-trimethylbenzoyl) phenylphosphinate, Phenyl(2,4,6-trimethylbenzoyl)phosphinic acid, glycerol ethoxylated trimester (Omnipol® TP from IGM Resins B.V.).

Examples of the halomethyltriazines based photoinitiators are 2-[2-(4-methoxy-phenyl)-vinyl]-4,6-bis-trichloromethyl [1,3,5]triazine, 2-(4-methoxy-phenyl)-4,6-bis-trichloromethyl [1,3,5]triazine, 2-(3,4-dimethoxyphenyl)-4,6-bis-trichloromethyl [1,3,5]triazine, 2-methyl-4,6-bis-trichloromethyl [1,3,5]triazine.

Cationic photoinitiators can be also used as the further photoinitiators (e), when the photocurable compositions according to the invention are used in hybrid systems (which in this connection mean mixtures of free-radically and cationically curing systems). Examples of suitable cationic photoinitiators are aromatic sulfonium, phosphonium or iodonium salts, as described e.g. in U.S. Pat. No. 4,950,581, or cyclopentadienylarene-iron(II) complex salts, e.g. (η⁶-isopropylbenzene)(η⁵-cyclopentadienyl) iron(II) hexafluorophosphate or photolatent acids based on oximes, as described, for example, in GB 2 348 644, U.S. Pat. Nos. 4,450,598, 4,136,055, WO 00/10972 and WO 00/26219.

The photocuring composition according to the invention may also comprise conventional additives, from 0 to 10% based on the total content of the composition. Additives (f) can be, for example, thermal initiators, binders, stabilizers, and mixture thereof.

The choice of additives is governed by the field of use in question and the properties desired for that field. The additives (f) described above are known in the art and are accordingly used in the amounts conventionally used in the art.

For instance, especially in the case of pigmented compositions, the composition may also comprise, as additional additive (f), a thermal initiator, a compound that forms free radicals when heated, e.g. an azo compounds, such as 2,2′-azobis(4-methoxy-2,4-dimethylvaleronitrile), a triazene, diazosulfide, pentazadiene or a peroxy compound, for example a hydroperoxide or peroxycarbonate, e.g. tert-butyl hydroperoxide, as described e.g. in EP 245 639.

Binders may also be added to the photocurable composition of the invention. The addition of binders is particularly advantageous when the photocurable compounds are liquid or viscous substances. The amount of binder may be, for example, from 5 to 60% by weight, preferably from 10 to 50% by weight, based on the total content of the composition, excluding possible water and solvents. The choice of binder is made in accordance with the field of use and the properties required therefor, such as developability in aqueous and organic solvent systems, adhesion to substrates and sensitivity to oxygen. Suitable binders are, for example, polymers having a weight average molecular weight (Mw) of approximately from 5,000 Da to 2,000,000 Da, preferably from 10,000 Da to 1,000,000 Da. Illustrative examples are: homo- and copolymers of acrylates and methacrylates, e.g. copolymers of methyl methacrylate/ethyl acrylate/methacrylic acid, poly(methacrylic acid alkyl esters), poly(acrylic acid alkyl esters); cellulose esters and ethers, such as cellulose acetate, cellulose acetate butyrate, methylcellulose, ethylcellulose, polyvinylbutyral, polyvinylformal, cyclised rubber, polyethers such as polyethylene oxide, polypropylene oxide, polytetrahydrofuran, polystyrene, polycarbonates, polyurethanes, chlorinated polyolefins, e.g. polyvinyl chloride, co-polymers of vinyl chloride/vinylidene chloride, co-polymers of vinylidene chloride with acrylonitrile, methyl methacrylate and vinyl acetate, polyvinyl acetate, co-poly (ethylene/vinyl acetate), polymers such as polycaprolactam and poly(hexamethylene adipamide), polyesters such as poly(ethylene glycol terephthalate) and poly(hexamethylene glycol succinate).

Suitable stabilizers are, for example, thermal inhibitors, such as hydroquinone, hydroquinone derivatives, p-methoxyphenol, β-benzol or sterically hindered phenols, e.g. 2,6-di(tert-butyl)-p-cresol, which prevent premature polymerization. In order to increase dark storage stability it is possible to use, for example, copper compounds, such as copper naphthenate, stearate or octoate, phosphorus compounds, for example triphenylphosphine, tributylphosphine, triethyl phosphite, triphenyl phosphite or tribenzyl phosphite, quaternary ammonium compounds, e.g. tetramethylammonium chloride or trimethylbenzylammonium chloride, or hydroxylamine derivatives, e.g. N,N-diethylhydroxylamine. For the purpose of excluding atmospheric oxygen during polymerization it is possible to add paraffin or similar wax-like substances which, being insoluble in the polymer, migrate to the surface at the beginning of the polymerization and form a transparent surface layer which prevents air from entering.

It is also possible to add a light stabilizer, such as UV absorbers, e.g. hydroxyphenylbenzotriazole, hydroxyphenylbenzophenone, oxalic acid amide or hydroxyphenyl-s-triazine type. Such components can be used on their own or in the form of mixtures, with or without the use of sterically hindered amines (HALS).

The photocurable compositions according to the invention may also comprise, as further additives (f), photoreducible dyes, e.g. a xanthene, benzoxanthene, benzothioxanthene, thiazine, pyronin, porphyrin or acridine dye, and/or radiation cleavable trihalomethyl compounds. These compounds are described, for example, in EP445624.

Further customary additives (f) are, depending upon the intended use, optical brighteners, fillers, pigments, both white and colored pigments, colorants, antistatics, wetting agents or flow improvers. Additives conventionally used in the art, e.g. antistatics, flow improvers and adhesion enhancers, can also be used.

In addition to the above components, other components may be present in the composition of the invention.

It is also possible for chain-transfer reagents conventionally used in the art to be added to the compositions according to the invention. Examples are mercaptans, amines and benzothiazole.

The composition of the invention may also comprise colorants and/or colored pigments. Depending upon the intended use, both inorganic and organic pigments may be used. Such additives are well known to the person skilled in the art; some examples are carbon black, iron oxides, such as iron oxide yellow, iron oxide red, chromium yellow, chromium green, nickel titanium yellow, ultramarine blue, cobalt blue, bismuth vanadate, cadmium yellow and cadmium red. Examples of organic pigments are mono- or bis-azo pigments, and also metal complexes thereof, phthalocyanine pigments, polycyclic pigments, e.g. perylene, anthraquinone, thioindigo, quinacridone or triphenylmethane pigments, and also diketo-pyrrolo-pyrrole, isoindolinone, e.g. tetrachloroisoindolinone, isoindoline, dioxazine, benzimidazolone and quinophthalone pigments. The pigments may be used in the formulations on their own or in admixture.

Depending upon the intended use, the pigments can be added to the formulations in amounts conventionally used in the art, for example in an amount from 0.1 to 30% by weight or from 10 to 25% by weight, based on the total weight of the composition.

The composition may also comprise, for example, organic colorants of an extremely wide variety of classes. Examples are azo dyes, methine dyes, anthraquinone dyes and metal complex dyes. Usual concentrations are, for example, from 0.1 to 20% wt, especially from 1 to 5% wt, based on the total weight of the composition.

The photocurable compositions of the invention may comprise water.

The photocurable compositions of the invention are suitable for various purposes, for example as a printing ink, such as screen printing inks, flexographic printing inks, offset printing inks and inkjet printing inks, as clearcoats, as colored coats, for example for wood or metal, as powder coatings, as coating materials inter alia for paper, wood, metal or plastics, as daylight-curable paints for marking structures and roads, for photographic reproduction processes, for holographic recording materials, for image-recording processes or in the production of printing plates that can be developed using organic solvents or using aqueous-alkaline media, for the production of masks for screen printing, as dental filling compounds, as adhesives, as pressure-sensitive adhesives, as laminating resins, as photoresists, e.g. galvanoresists, as etch resists or permanent resists, both liquid and dry films, as photostructurable dielectrics, and as solder masks for electronic circuits, as resists in the production of color filters for any type of display screen or in the creation of structures during the manufacture of plasma displays and electroluminescent displays, in the production of optical switches, optical gratings (interference gratings), in the manufacture of three-dimensional articles by bulk curing (UV curing in transparent moulds) or according to the stereolithography process, as described, for example, in U.S. Pat. No. 4,575,330, in the manufacture of composite materials (e.g. styrene polyesters which may include glass fibers and/or other fibers and other adjuvants) and other methods of printing in three dimensions well-known to one skilled in the art, in the coating or sealing of electronic components or as coatings for optical fibers.

The photocurable compositions of the invention are also suitable for the production of optical lenses, e.g. contact lenses or Fresnel lenses, in the manufacture of medical apparatus, aids or implants, in dry film paints.

The photocurable compositions of the invention are also suitable for the preparation of gels having thermotropic properties. Such gels are described for example in DE 19700064 and EP 678534.

An article comprising a compound of formula (I), or comprising a photocurable composition of the invention, represents a further subject-matter of the invention.

The compounds and compositions according to the invention may also be used as free-radical photoinitiators or photoinitiating systems for radiation-curable powder coatings.

The photocurable compositions according to the invention are suitable, for example, as coating materials for all kinds of substrate, for example wood, textiles, paper, ceramics, glass, plastics, such as polyesters, polyethylene terephthalate, polyolefins and cellulose acetate, especially in the form of films, and also metals, such as Al, Cu, Ni, Fe, Zn, Mg or Co and GaAs, Si or SiO₂, to which a protective layer is to be applied or an image is to be applied e.g. by imagewise exposure.

A large number of the most varied kinds of light source may be used in the process according to the invention, the light source emitting at wavelengths from approximately 200 nm to approximately 800 nm. Both point sources and planiform radiators (lamp carpets) are suitable. Examples are: carbon arc lamps, xenon arc lamps, medium pressure, high pressure and low pressure mercury arc radiators, doped, where appropriate, with metal halides (metal halide lamps), microwave-excited metal vapour lamps, excimer lamps, superactinic fluorescent tubes, fluorescent lamps, argon incandescent lamps, flash lamps, photographic floodlight lamps, light-emitting diodes (LED), electron beams, X-rays and lasers.

According to one embodiment, said light source comprises UV light in at least one of the UVA, UVB and UVC ranges.

According to a preferred embodiment, said light source is a LED source, particularly preferred are LED light source emitting at wavelengths comprised between 365 nm and 420 nm, more preferably at 365 nm, 385 nm and 395 nm.

According to the invention the distance between the lamp and the substrate to be exposed may vary according to the intended use and the type and strength of the lamp, e.g. from 0.1 cm to 150 cm, preferably from 1 cm to 50 cm.

Said photopolymerizable composition may also be applied over a substrate already comprising a coated or printed layer. Said photopolymerizable composition may, after photopolymerization with said light source, be overprinted or overcoated with one or more compositions suitable for printing or coating.

The article obtained by applying said photopolymerizable composition to said substrate by said means of coating or printing, and photopolymerizing by said light source, with or without further elaboration of the article by further coating or printing, represents a further subject-matter of this invention.

As said, we surprisingly found that compounds of formula (I) have a very high reactivity under UVA, UVB and UVC wavelength maintaining a low post cure yellowing compared to that of the prior art. The new compounds showed their great improvements under LED and Hg lamps in clear and pigmented systems.

The invention is illustrated in detail below by the following examples, which are illustrative and not limiting.

In case of inconsistencies between the chemical name and the chemical structure herein indicated, the chemical structure prevails.

### EXPERIMENTAL SECTION

In all the present specification, in case of inconsistences between a chemical name and a chemical formula, the latter prevails.

Compounds of Examples 14 to 17 are mixtures of compounds obtained by the reaction of the indicated starting materials.

¹H NMR spectra were recorded with a Bruker Ascend 300 MHz NMR Spectrometer.

### Example 1

### Synthesis of

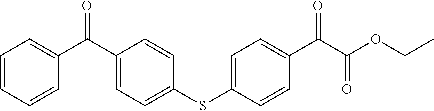 [SMILES: CCOC(=O)C(=O)c1ccc(Sc2ccc(C(=O)c3ccccc3)cc2)cc1]

To an ice-cooled mixture of 10.00 g (53.686 mmoles) of Diphenyl sulfide and 7.92 g (56.342 mmoles) of Benzoyl chloride in 120 mL of dichloromethane, 7.87 g (59.022 mmoles) of anhydrous aluminum chloride were cautiously added in portions. After stirring for 1.5 hours at room temperature, the reaction was cooled again and 9.53 g (69.802 mmoles) of Ethyl chlorooxoacetate and 10.88 g (81.596 mmoles) of anhydrous aluminum chloride were cautiously added in sequence. After stirring for 1.5 hours at room temperature, the reaction was cooled again and additional 1.47 g (10.767 mmoles) of Ethyl chlorooxoacetate and 1.65 g (12.374 mmoles) of anhydrous aluminum chloride were cautiously added in sequence. Then the reaction was stirred at room temperature for further 1.5 hours and poured into 400 mL of ice/water. The mixture was extracted with dichloromethane and the organic layer was separated, washed again with water and brine, dried over anhydrous sodium sulfate, filtered and the solvent removed by distillation under vacuum obtaining the crude. The crude product was purified by crystallization from ethanol obtaining 15.70 g of an off-white solid (yield 75%).

¹H-NMR (DMSO-d₆, δ ppm): 1.33 (t, 3H), 4.42 (q, 2H), 7.51 (d, 2H), 7.54-7.74 (m, 5H), 7.76-7.82 (m, 4H), 7.97 (d, 2H).

### Example 2

### Synthesis of

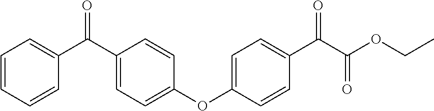 [SMILES: CCOC(=O)C(=O)c1ccc(Oc2ccc(C(=O)c3ccccc3)cc2)cc1]

To an ice-cooled mixture of 5.00 g (29.375 mmoles) of Diphenyl ether and 4.33 g (30.803 mmoles) of Benzoyl chloride in 60 mL of dichloromethane, 4.31 g (32.323 mmoles) of anhydrous aluminum chloride were cautiously added in portions. After stirring for 1.5 hours at room temperature, the reaction was cooled again and 5.21 g (38.160 mmoles) of Ethyl chlorooxoacetate and 5.95 g (44.623 mmoles) of anhydrous aluminum chloride were cautiously added in sequence. After stirring for 1.5 hours at room temperature, the reaction was cooled again and additional 0.80 g (5.860 mmoles) of Ethyl chlorooxoacetate and 0.90 g (6.750 mmoles) of anhydrous aluminum chloride were cautiously added in sequence. Then the reaction was stirred at room temperature for further 1.5 hours and poured into 200 mL of ice/water. The mixture was extracted with dichloromethane and the organic layer was separated, washed again with water and brine, dried over anhydrous sodium sulfate, filtered and the solvent removed by distillation under vacuum obtaining the crude. The crude product was purified by crystallization from ethanol followed by flash column chromatography on silica gel (toluene/ethyl acetate 98:2) obtaining 7.97 g of a white solid (yield 72%).

¹H-NMR (DMSO-d₆, δ ppm): 1.34 (t, 3H), 4.42 (q, 2H), 7.30 (m, 4H), 7.58 (t, 2H), 7.67 (tt, 1H), 7.75 (dd, 2H), 7.86 (d, 2H), 8.06 (d, 2H).

### Example 3

### Synthesis of

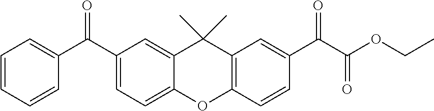 [SMILES: CCOC(=O)C(=O)c1ccc2c(c1)C(C)(C)c1cc(C(=O)c3ccccc3)ccc1O2]

To an ice-cooled mixture of 10.00 g (47.556 mmoles) of 9,9-Dimethylxanthene and 7.02 g (49.940 mmoles) of Benzoyl chloride in 120 mL of dichloromethane, 6.98 g (52.347 mmoles) of anhydrous aluminum chloride were cautiously added in portions. After stirring for 1.5 hours at room temperature, the reaction was cooled again and 8.44 g (61.818 mmoles) of Ethyl chlorooxoacetate and 9.64 g (72.297 mmoles) of anhydrous aluminum chloride were cautiously added in sequence. After stirring for 1.5 hours at room temperature, the reaction was cooled again and additional 1.30 g (9.522 mmoles) of Ethyl chlorooxoacetate and 1.46 g (10.949 mmoles) of anhydrous aluminum chloride were cautiously added in sequence. Then the reaction was stirred at room temperature for further 1.5 hours and poured into 400 mL of ice/water. The mixture was extracted with dichloromethane and the organic layer was separated, washed again with water and brine, dried over anhydrous sodium sulfate, filtered and the solvent removed by distillation under vacuum obtaining the crude. The crude product was purified by flash column chromatography on silica gel (toluene/ethyl acetate 98:2) obtaining 16.74 g of a yellow oil (yield 85%).

¹H-NMR (DMSO-d₆, δ ppm): 1.33 (t, 3H), 1.64 (s, 6H), 4.43 (q, 2H), 7.26 (d, 1H), 7.31 (d, 1H), 7.54 (t, 2H), 7.62-7.75 (m, 4H), 7.88 (dd, 1H), 7.99 (d, 1H), 8.18 (d, 1H).

### Example 4

### Synthesis of

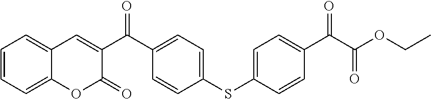 [SMILES: CCOC(=O)C(=O)c1ccc(Sc2ccc(C(=O)c3cc4ccccc4oc3=O)cc2)cc1]

### Step a) Synthesis of

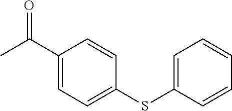 [SMILES: CC(=O)c1ccc(Sc2ccccc2)cc1]

To an ice-cooled mixture of 5.00 g (26.843 mmoles) of Diphenyl sulfide and 2.21 g (28.153 mmoles) of acetyl chloride in 50 mL of dichloromethane, 3.94 g (29.549 mmoles) of anhydrous aluminum chloride were cautiously added in portions. After stirring for 1.5 hours at room temperature, the reaction was poured into 200 mL of ice/water. The mixture was extracted with dichloromethane and the organic layer was separated, washed again with water and brine, dried over anhydrous sodium sulfate, filtered and the solvent removed by distillation under vacuum obtaining 5.97 g of an off-white solid (yield 97%).

¹H-NMR (DMSO-d₆, δ ppm): 2.53 (s, 3H), 7.25 (d, 2H), 7.48 (m, 5H), 7.87 (d, 2H)

### Step b) Synthesis of

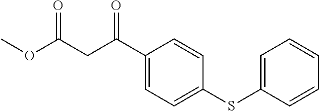 [SMILES: COC(=O)CC(=O)c1ccc(Sc2ccccc2)cc1]

4.73 g (26.268 mmoles) of sodium methoxide 30% w/w in methanol were slowly added portionwise in 10 minutes under stirring at 90° C. to a mixture of 5.00 g (21.900 mmoles) of the compound prepared in the previous step and 19.73 g (219.028 mmoles) of dimethyl carbonate in 18.4 mL of toluene. The mixture was stirred at 90° C. for 1 hour eliminating methanol by distillation. After completion of the reaction, the mixture was allowed to cool, poured into 200 mL of 3M hydrochloric acid and extracted with ethyl acetate. The organic layer was washed with water and brine, dried over anhydrous sodium sulfate, filtered and the solvent removed by distillation under vacuum obtaining 6.25 g of a yellow oil (yield 100%).

¹H-NMR (DMSO-d₆, δ ppm): 3.65 (s, 3H), 4.15 (s, 2H), 7.25 (d, 2H), 7.51 (m, 5H), 7.87 (d, 2H).

### Step c) Synthesis of

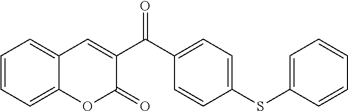 [SMILES: O=C(c1ccc(Sc2ccccc2)cc1)c1cc2ccccc2oc1=O]

0.178 g (2.090 mmoles) of piperidine were added to a mixture of 6.00 g (20.953 mmoles) of the compound prepared in the previous step and 2.56 g (20.963 mmoles) of salicylaldehyde in 80 mL of ethanol. The mixture was stirred for 1 hour under reflux, then cooled to room temperature. The reaction product was recovered by filtration obtaining 6.63 g of a yellow solid (yield 88%).

¹H-NMR (DMSO-d₆, δ ppm): 7.23 (d, 2H), 7.42 (td, 1H), 7.47-7.58 (m, 6H), 7.73 (m, 1H), 7.83-7.88 (m, 3H), 8.40 (s, 1H).

### Step d)

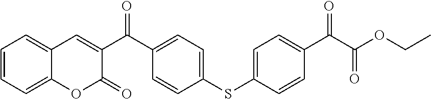 [SMILES: CCOC(=O)C(=O)c1ccc(Sc2ccc(C(=O)c3cc4ccccc4oc3=O)cc2)cc1]

To an ice-cooled mixture of 5.00 g (13.951 mmoles) of the compound prepared in the previous step and 2.48 g (18.165 mmoles) of Ethyl chlorooxoacetate in 100 mL of dichloromethane, 6.55 g (49.123 mmoles) of anhydrous aluminum chloride were cautiously added in portions. After stirring for 1.5 hours at room temperature, the reaction was poured into 200 mL of ice/water. The mixture was extracted with dichloromethane and the organic layer was separated, washed again with water and brine, dried over anhydrous sodium sulfate, filtered and the solvent removed by distillation under vacuum obtaining the crude. The crude product was taken up in 120 mL of ethanol and washed under reflux and vigorous stirring for 30 minutes, then cooled to room temperature. The reaction product was recovered by filtration obtaining 6.00 g of a canary yellow solid (yield 94%).

¹H-NMR (DMSO-d₆, δ ppm): 1.33 (t, 3H), 4.41 (q, 2H), 7.43 (td, 1H), 7.48-7.60 (m, 5H), 7.75 (m, 1H), 7.88 (dd, 1H), 7.95-8.01 (m, 4H), 8.47 (s, 1H).

### Example 5

### Synthesis of

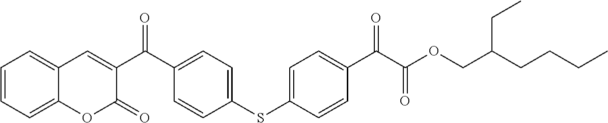 [SMILES: CCCCC(CC)COC(=O)C(=O)c1ccc(Sc2ccc(C(=O)c3cc4ccccc4oc3=O)cc2)cc1]

0.53 g (1.087 mmoles) of zirconium(IV) acetylacetonate were added under stirring to a warm solution of 5.00 g (10.906 mmoles) of Example 4 and 4.26 g (32.711 mmoles) of 2-ethyl-1-hexanol in 40 mL of toluene. The reaction mixture was stirred at 110° C. for 2 hours eliminating ethanol by distillation. Progress of the reaction was monitored by TLC. After completion of the reaction, the mixture was allowed to cool, diluted with ethyl acetate and washed in sequence with 50 mL (×2) of 3M hydrochloric acid, water and brine, respectively. The organic layer was separated, dried over anhydrous sodium sulfate, filtered and the solvent removed by distillation under vacuum obtaining the crude. The crude product was purified by crystallization from ethanol obtaining 5.09 g of a canary yellow solid (yield 86%).

¹H-NMR (DMSO-d₆, δ ppm): 0.85 (m, 6H), 1.32 (m, 8H), 1.66 (m, 1H), 4.30 (d, 2H), 7.41 (td, 1H), 7.48-7.60 (m, 5H), 7.75 (m, 1H), 7.88 (dd, 1H), 7.93-8.00 (m, 4H), 8.47 (s, 1H).

### Example 6

### Synthesis of

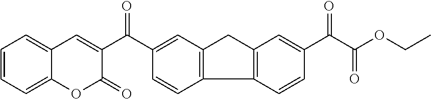 [SMILES: CCOC(=O)C(=O)c1ccc2c(c1)Cc1cc(C(=O)c3cc4ccccc4oc3=O)ccc1-2]

### Step a) Synthesis of

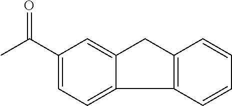 [SMILES: CC(=O)c1ccc2c(c1)Cc1ccccc1-2]

To an ice-cooled mixture of 5.00 g (30.081 mmoles) of fluorene and 2.48 g (31.592 mmoles) of acetyl chloride in 50 mL of dichloromethane, 4.41 g (33.073 mmoles) of anhydrous aluminum chloride were cautiously added in portions. After stirring for 1.5 hours at room temperature, the reaction was cooled again and additional 0.118 g (1.503 mmoles) of acetyl chloride and 0.240 g (1.800 mmoles) of anhydrous aluminum chloride were cautiously added in sequence. Then the reaction was stirred at room temperature for further 45 minutes and poured into 200 mL of ice/water. The mixture was extracted with dichloromethane and the organic layer was separated, washed again with water and brine, dried over anhydrous sodium sulfate, filtered and the solvent removed by distillation under vacuum obtaining 6.25 g of an off-white solid (yield 100%).

¹H-NMR (DMSO-d₆, δ ppm): 2.63 (s, 3H), 4.01 (s, 2H), 7.41 (m, 2H), 7.64 (m, 1H), 8.00 (m, 3H), 8.17 (s, 1H).

### Step b) Synthesis of

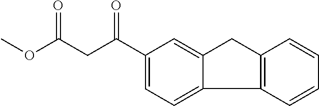 [SMILES: COC(=O)CC(=O)c1ccc2c(c1)Cc1ccccc1-2]

5.19 g (28.823 mmoles) of sodium methoxide 30% w/w in methanol were slowly added portionwise in 10 minutes under stirring at 90° C. to a mixture of 5.00 g (24.008 mmoles) of the compound of the previous step and 21.63 g (240.120 mmoles) of dimethyl carbonate in 20.2 mL of toluene. The mixture was stirred at 90° C. for 1 hour eliminating methanol by distillation. After completion of the reaction, the mixture was allowed to cool, poured into 200 mL of 3M hydrochloric acid and extracted with ethyl acetate. The organic layer was washed with water and brine, dried over anhydrous sodium sulfate, filtered and the solvent removed by distillation under vacuum obtaining 6.27 g of a yellow oil (yield 98%).

¹H-NMR (DMSO-d₆, δ ppm): 3.68 (s, 3H), 4.02 (s, 2H), 4.26 (s, 2H), 7.43 (m, 2H), 7.65 (m, 1H), 8.01 (m, 3H), 8.18 (s, 1H).

### Step c) Synthesis of

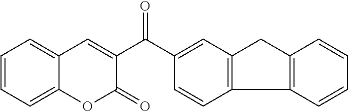 [SMILES: O=C(c1ccc2c(c1)Cc1ccccc1-2)c1cc2ccccc2oc1=O]

0.192 g (2.255 mmoles) of piperidine were added to a mixture of 6.00 g (22.531 mmoles) of the compound of the previous step and 2.75 g (22.519 mmoles) of salicylaldehyde in 120 mL of ethanol. The mixture was stirred for 1 hour under reflux, then cooled to room temperature. The reaction product was recovered by filtration obtaining 5.96 g of a canary yellow solid (yield 78%).

¹H-NMR (DMSO-d₆, δ ppm): 4.02 (s, 2H), 7.40-7.48 (m, 3H), 7.51 (d, 1H), 7.66 (m, 1H), 7.75 (m, 1H), 7.88 (dd, 1H), 7.99-8.08 (m, 3H), 8.18 (s, 1H), 8.43 (s, 1H).

### Step d) Synthesis of

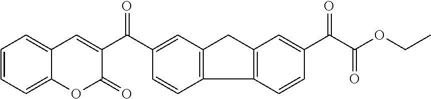 [SMILES: CCOC(=O)C(=O)c1ccc2c(c1)Cc1cc(C(=O)c3cc4ccccc4oc3=O)ccc1-2]

To an ice-cooled mixture of 5.00 g (14.777 mmoles) of the compound of the previous step and 2.62 g (19.190 mmoles) of Ethyl chlorooxoacetate in 100 mL of dichloromethane, 6.94 g (52.047 mmoles) of anhydrous aluminum chloride were cautiously added in portions. After stirring for 1.5 hours at room temperature, the reaction was cooled again and additional 0.404 g (2.959 mmoles) of Ethyl chlorooxacetate and 0.453 g (3.397 mmoles) of anhydrous aluminum chloride were cautiously added in sequence. Then the reaction was stirred at room temperature for further 1 hour and poured into 200 mL of ice/water. The mixture was extracted with dichloromethane and the organic layer was separated, washed again with water and brine, dried over anhydrous sodium sulfate, filtered and the solvent removed by distillation under vacuum obtaining the crude. The crude product was taken up in 70 mL of toluene and washed under reflux and vigorous stirring for 30 minutes, then cooled to room temperature. The reaction product was recovered by filtration obtaining 4.66 g of a yellow solid (yield 72%).

¹H-NMR (DMSO-d₆, δ ppm): 1.37 (t, 3H), 4.15 (s, 2H), 4.48 (q, 2H), 7.45 (td, 1H), 7.53 (d, 1H), 7.75 (m, 1H), 7.88 (dd, 1H), 8.05 (m, 2H), 8.21-8.29 (m, 4H), 8.47 (s, 1H).

### Example 7

### Synthesis of

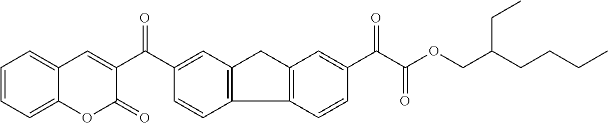 [SMILES: CCCCC(CC)COC(=O)C(=O)c1ccc2c(c1)Cc1cc(C(=O)c3cc4ccccc4oc3=O)ccc1-2]

0.501 g (1.027 mmoles) of zirconium(IV) acetylacetonate were added under stirring to a warm solution of 4.50 g (10.264 mmoles) of Example 6 and 4.01 g (30.792 mmoles) of 2-ethyl-1-hexanol in 35 mL of toluene. The reaction mixture was stirred at 110° C. for 2.5 hours eliminating ethanol by distillation. Progress of the reaction was monitored by TLC. After completion of the reaction, the mixture was allowed to cool, diluted with dichloromethane and washed in sequence with 50 mL (×2) of 3M hydrochloric acid, water and brine, respectively. The organic layer was separated, dried over anhydrous sodium sulfate, filtered and the solvent removed by distillation under vacuum obtaining the crude. The crude product was taken up in 100 mL of ethanol and washed under reflux and vigorous stirring for 30 minutes, then cooled to room temperature. The reaction product was recovered by filtration obtaining 4.45 g of a yellow solid (yield 83%).

¹H-NMR (DMSO-d₆, δ ppm): 0.88 (m, 6H), 1.33 (m, 8H), 1.70 (m, 1H), 4.15 (s, 2H), 4.37 (d, 2H), 7.45 (td, 1H), 7.53 (d, 1H), 7.75 (m, 1H), 7.88 (dd, 1H), 8.05 (td, 2H), 8.22-8.30 (m, 4H), 8.47 (s, 1H).

### Example 8

### Synthesis of

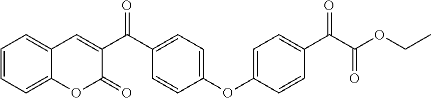 [SMILES: CCOC(=O)C(=O)c1ccc(Oc2ccc(C(=O)c3cc4ccccc4oc3=O)cc2)cc1]

### Step a) Synthesis of

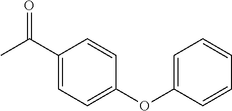 [SMILES: CC(=O)c1ccc(Oc2ccccc2)cc1]

To an ice-cooled mixture of 5.00 g (29.375 mmoles) of Diphenyl ether and 2.42 g (30.828 mmoles) of acetyl chloride in 50 mL of dichloromethane, 4.31 g (32.323 mmoles) of anhydrous aluminum chloride were cautiously added in portions. After stirring for 1.5 hours at room temperature, the reaction was poured into 200 mL of ice/water. The mixture was extracted with dichloromethane and the organic layer was separated, washed again with water and brine, dried over anhydrous sodium sulfate, filtered and the solvent removed by distillation under vacuum obtaining 6.12 g of a white solid (yield 98%).

¹H-NMR (DMSO-d₆, δ ppm): 2.55 (s, 3H), 7.05 (d, 2H), 7.12 (d, 2H), 7.25 (t, 1H), 7.47 (t, 2H), 7.99 (d, 2H).

### Step b) Synthesis of

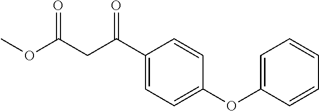 [SMILES: COC(=O)CC(=O)c1ccc(Oc2ccccc2)cc1]

5.09 g (28.267 mmoles) of sodium methoxide 30% w/w in methanol were slowly added portionwise in 10 minutes under stirring at 90° C. to a mixture of 5.00 g (23.557 mmoles) of the compound of the previous step and 21.22 g (235.568 mmoles) of dimethyl carbonate in 19.8 mL of toluene. The mixture was stirred at 90° C. for 1 hour eliminating methanol by distillation. After completion of the reaction, the mixture was allowed to cool, poured into 200 mL of 3M hydrochloric acid and extracted with ethyl acetate. The organic layer was washed with water and brine, dried over anhydrous sodium sulfate, filtered and the solvent removed by distillation under vacuum obtaining 6.34 g of a yellow oil (yield 100%).

¹H-NMR (DMSO-d₆, δ ppm): 3.66 (s, 3H), 4.16 (s, 2H), 7.05 (d, 2H), 7.13 (d, 2H), 7.28 (t, 1H), 7.47 (t, 2H), 7.99 (d, 2H).

### Step c) Synthesis of

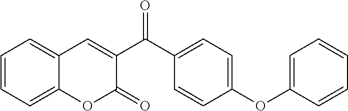 [SMILES: O=C(c1ccc(Oc2ccccc2)cc1)c1cc2ccccc2oc1=O]

0.189 g (2.220 mmoles) of piperidine were added to a mixture of 6.00 g (22.199 mmoles) of the compound of the previous step and 2.71 g (22.191 mmoles) of salicylaldehyde in 80 mL of ethanol. The mixture was stirred for 1 hour under reflux, then cooled to room temperature. The reaction product was recovered by filtration obtaining 6.74 g of a white solid (yield 89%).

¹H-NMR (DMSO-d₆, δ ppm): 7.05 (d, 2H), 7.15 (d, 2H), 7.28 (t, 1H), 7.42-7.51 (m, 4H), 7.72 (m, 1H), 7.85 (dd, 1H), 7.99 (d, 2H), 8.40 (s, 1H).

### Step d) Synthesis of

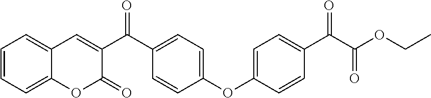 [SMILES: CCOC(=O)C(=O)c1ccc(Oc2ccc(C(=O)c3cc4ccccc4oc3=O)cc2)cc1]

To an ice-cooled mixture of 5.00 g (14.605 mmoles) of the compound of the previous step and 2.59 g (18.970 mmoles) of Ethyl chlorooxoacetate in 100 mL of dichloromethane, 6.85 g (51.372 mmoles) of anhydrous aluminum chloride were cautiously added in portions. After stirring for 1.5 hours at room temperature, the reaction was poured into 200 mL of ice/water. The mixture was extracted with dichloromethane and the organic layer was separated, washed again with water and brine, dried over anhydrous sodium sulfate, filtered and the solvent removed by distillation under vacuum obtaining the crude. The crude product was taken up in 100 mL of ethanol and washed under reflux and vigorous stirring for 30 minutes, then cooled to room temperature. The reaction product was recovered by filtration obtaining 5.96 g of a white solid (yield 92%).

¹H-NMR (DMSO-d₆, δ ppm): 1.34 (t, 3H), 4.42 (q, 2H), 7.28 (m, 4H), 7.44 (td, 1H), 7.50 (d, 1H), 7.75 (m, 1H), 7.88 (dd, 1H), 8.06 (m, 4H), 8.44 (s, 1H).

### Example 9

### Synthesis of

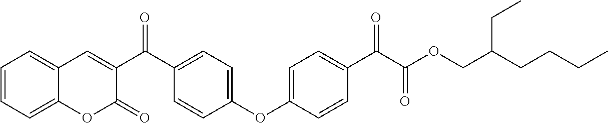 [SMILES: CCCCC(CC)COC(=O)C(=O)c1ccc(Oc2ccc(C(=O)c3cc4ccccc4oc3=O)cc2)cc1]

0.646 g (1.325 mmoles) of zirconium(IV) acetylacetonate were added under stirring to a warm solution of 5.86 g (13.245 mmoles) of Example 8 and 5.17 g (39.699 mmoles) of 2-ethyl-1-hexanol in 46 mL of toluene. The reaction mixture was stirred at 110° C. for 2 hours eliminating ethanol by distillation. Progress of the reaction was monitored by TLC. After completion of the reaction, the mixture was allowed to cool, diluted with ethyl acetate and washed in sequence with 50 mL (×2) of 3M hydrochloric acid, water and brine, respectively. The organic layer was separated, dried over anhydrous sodium sulfate, filtered and the solvent removed by distillation under vacuum obtaining the crude. The crude product was purified by crystallization from ethanol obtaining 5.63 g of a white solid (yield 81%).

¹H-NMR (DMSO-d₆, δ ppm): 0.86 (m, 6H), 1.33 (m, 8H), 1.70 (m, 1H), 4.30 (d, 2H), 7.28 (m, 4H), 7.44 (td, 1H), 7.51 (d, 1H), 7.75 (m, 1H), 7.88 (dd, 1H), 8.06 (m, 4H), 8.44 (s, 1H).

### Example 10

### Synthesis of

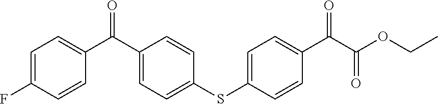 [SMILES: CCOC(=O)C(=O)c1ccc(Sc2ccc(C(=O)c3ccc(F)cc3)cc2)cc1]

To an ice-cooled mixture of 5.00 g (26.843 mmoles) of Diphenyl sulfide and 4.47 g (28.191 mmoles) of 4-Fluorobenzoyl chloride in 60 mL of dichloromethane, 3.94 g (29.549 mmoles) of anhydrous aluminum chloride were cautiously added in portions. After stirring for 1.5 hours at room temperature, the reaction was cooled again and 5.50 g (40.284 mmoles) of Ethyl chlorooxoacetate and 6.26 g (46.948 mmoles) of anhydrous aluminum chloride were cautiously added in sequence. After stirring for 1.5 hours at room temperature, the reaction was cooled again and additional 3.57 g (26.774 mmoles) of anhydrous aluminum chloride were cautiously added. Then the reaction was stirred at room temperature for further 30 minutes and poured into 200 mL of ice/water. The mixture was extracted with dichloromethane and the organic layer was separated, washed again with water and brine, dried over anhydrous sodium sulfate, filtered and the solvent removed by distillation under vacuum obtaining the crude. The crude product was taken up in 110 mL of ethanol and washed under reflux and vigorous stirring for 30 minutes, then cooled to room temperature. The reaction product was recovered by filtration obtaining 9.80 g of a white solid (yield 89%).

¹H-NMR (DMSO-d₆, δ ppm): 1.33 (t, 3H), 4.42 (q, 2H), 7.40 (t, 2H), 7.52 (d, 2H), 7.64 (d, 2H), 7.78 (d, 2H), 7.85 (m, 2H), 7.95 (d, 2H).

### Example 11

### Synthesis of

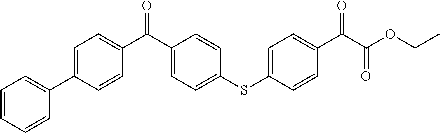 [SMILES: CCOC(=O)C(=O)c1ccc(Sc2ccc(C(=O)c3ccc(-c4ccccc4)cc3)cc2)cc1]

To an ice-cooled mixture of 5.00 g (26.843 mmoles) of Diphenyl sulfide and 6.11 g (28.201 mmoles) of Biphenyl-4-carbonyl chloride in 60 mL of dichloromethane, 3.94 g (29.549 mmoles) of anhydrous aluminum chloride were cautiously added in portions. After stirring for 1.5 hours at room temperature, the reaction was cooled again and additional 0.29 g (1.339 mmoles) of Biphenyl-4-carbonyl chloride and 0.21 g (1.575 mmoles) of anhydrous aluminum chloride were cautiously added in sequence. After stirring at room temperature for 30 minutes, the reaction was cooled again and 4.76 g (34.864 mmoles) of Ethyl chlorooxoacetate and 8.23 g (61.722 mmoles) of anhydrous aluminum chloride were cautiously added in sequence. After stirring at room temperature for 1.5 hours, the reaction was poured into 200 mL of ice/water. The mixture was extracted with dichloromethane and the organic layer was separated, washed again with water and brine, dried over anhydrous sodium sulfate, filtered and the solvent removed by distillation under vacuum obtaining the crude. The crude product was taken up in 110 mL of ethanol and washed under reflux and vigorous stirring for 30 minutes, then cooled to room temperature. The reaction product was recovered by filtration and the solid purified by crystallization from toluene obtaining 8.90 g of a white solid (yield 71%).

¹H-NMR (DMSO-d₆, δ ppm): 1.33 (t, 3H), 4.42 (q, 2H), 7.43-57 (m, 5H), 7.66 (d, 2H), 7.78 (d, 2H) 7.83-7.88 (m, 6H), 7.96 (d, 2H).

### Example 12

### Synthesis of

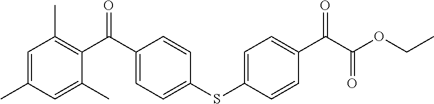 [SMILES: CCOC(=O)C(=O)c1ccc(Sc2ccc(C(=O)c3c(C)cc(C)cc3C)cc2)cc1]

To an ice-cooled mixture of 5.00 g (26.843 mmoles) of Diphenyl sulfide and 5.15 g (28.196 mmoles) of 2,4,6-trimethylbenzoyl chloride in 60 mL of dichloromethane, 3.94 g (29.549 mmoles) of anhydrous aluminum chloride were cautiously added in portions. After stirring for 2 hours at room temperature, the reaction was cooled again and additional 0.49 g (2.683 mmoles) of 2,4,6-trimethylbenzoyl chloride and 0.54 g (4.050 mmoles) of anhydrous aluminum chloride were cautiously added in sequence. After stirring for 1.5 hours at room temperature, the reaction was cooled again and additional 0.49 g (2.683 mmoles) of 2,4,6-trimethylbenzoyl chloride and 0.54 g (4.050 mmoles) of anhydrous aluminum chloride were cautiously added in sequence. After stirring at room temperature for 2 hours, the reaction was heated to reflux and stirred for further 45 minutes, then cooled again and 4.76 g (34.864 mmoles) of Ethyl chlorooxoacetate and 8.23 g (61.722 mmoles) of anhydrous aluminum chloride were cautiously added in sequence. After stirring at room temperature for 1.5 hours, the reaction was poured into 200 mL of ice/water. The mixture was extracted with dichloromethane and the organic layer was separated, washed again with water and brine, dried over anhydrous sodium sulfate, filtered and the solvent removed by distillation under vacuum obtaining the crude. The crude product was purified by flash column chromatography on silica gel (toluene/ethyl acetate 97.5:2.5) obtaining 7.12 g of a yellow oil (yield 61%).

¹H-NMR (DMSO-d₆, δ ppm): 1.33 (t, 3H), 2.00 (s, 6H), 2.30 (s, 3H), 4.40 (q, 2H), 6.96 (s, 2H), 7.51-7.58 (m, 4H), 7.70 (d, 2H), 7.96 (d, 2H).

### Example 13

### Synthesis of

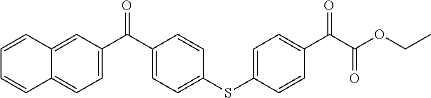 [SMILES: CCOC(=O)C(=O)c1ccc(Sc2ccc(C(=O)c3ccc4ccccc4c3)cc2)cc1]

To an ice-cooled mixture of 5.00 g (26.843 mmoles) of Diphenyl sulfide and 5.37 g (28.170 mmoles) of 2-benzoyl chloride in 60 mL of dichloromethane, 3.94 g (29.549 mmoles) of anhydrous aluminum chloride were cautiously added in portions. After stirring for 1.5 hours at room temperature, the reaction was cooled again and 4.76 g (34.864 mmoles) of Ethyl chlorooxoacetate and 8.23 g (61.722 mmoles) of anhydrous aluminum chloride were cautiously added in sequence. After stirring for 1.5 hours at room temperature, the reaction was poured into 200 mL of ice/water. The mixture was extracted with dichloromethane and the organic layer was separated, washed again with water and brine, dried over anhydrous sodium sulfate, filtered and the solvent removed by distillation under vacuum obtaining the crude. The crude product was purified by crystallization from 230 mL of ethanol obtaining 10.25 g of a white solid (yield 87%).

¹H-NMR (DMSO-d₆, δ ppm): 1.33 (t, 3H), 4.42 (q, 2H), 7.53 (d, 2H), 7.60-7.73 (m, 4H), 7.88 (m, 3H), 7.95 (d, 2H), 8.03-8.14 (m, 3H), 8.36 (d, 1H).

### Example 14

### Synthesis of

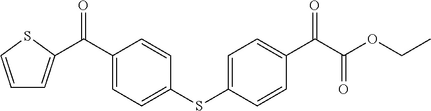 [SMILES: CCOC(=O)C(=O)c1ccc(Sc2ccc(C(=O)c3cccs3)cc2)cc1]

To an ice-cooled mixture of 5.00 g (26.843 mmoles) of Diphenyl sulfide and 4.13 g (28.174 mmoles) of Thiophene-2-carbonyl chloride in 60 mL of dichloromethane, 3.94 g (29.549 mmoles) of anhydrous aluminum chloride were cautiously added in portions. After stirring for 1.5 hours at room temperature, the reaction was cooled again and 4.76 g (34.864 mmoles) of Ethyl chlorooxoacetate and 8.23 g (61.722 mmoles) of anhydrous aluminum chloride were cautiously added in sequence. After stirring for 1.5 hours at room temperature, the reaction was poured into 200 mL of ice/water. The mixture was extracted with dichloromethane and the organic layer was separated, washed again with water and brine, dried over anhydrous sodium sulfate, filtered and the solvent removed by distillation under vacuum obtaining the crude. The crude product was purified by crystallization from ethanol obtaining 9.14 g of an off-white solid (yield 86%).

¹H-NMR (DMSO-d₆, δ ppm): 1.33 (t, 3H), 4.26 (q, 2H), 7.31 (m, 1H), 7.51 (d, 2H), 7.65 (d, 2H), 7.78 (dd, 1H), 7.90 (d, 2H), 7.97 (d, 2H), 8.15 (dd, 1H).

### Example 15

### Reaction Product of

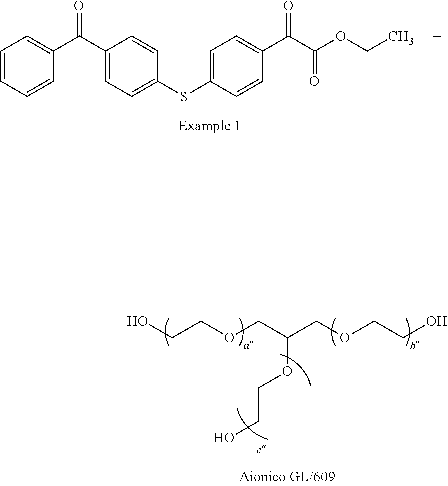

0.375 g (0.769 mmoles) of zirconium(IV) acetylacetonate were added under stirring to a warm solution of 3.00 g (7.683 mmoles) of Example 1 and 1.41 g (hydroxyl number 370 mg KOH/g) of Aionico GL/609 (purchased from Lamberti SpA) in 30 mL of toluene. Then the reaction mixture was stirred at 105-110° C. for 2 hours eliminating ethanol by distillation. Progress of the reaction was monitored by TLC (toluene/ethyl acetate 95:5). Then additional 0.061 g of Aionico GL/609 and 0.187 g (0.383 mmoles) of zirconium(IV) acetylacetonate were added and the reaction mixture was stirred at 105-110° C. for further 2.5 hours eliminating ethanol by distillation. Then additional 0.061 g of Aionico GL/609 and 0.187 g (0.383 mmoles) of zirconium(IV) acetylacetonate were added and the reaction mixture was stirred at 105-110° C. for further 4 hours eliminating ethanol by distillation. After completion of the reaction, the mixture was allowed to cool, diluted with ethyl acetate and washed in sequence with 50 mL (×2) of 1.5M hydrochloric acid, water and brine, respectively, dried over anhydrous sodium sulfate, filtered and the solvent removed by distillation under vacuum obtaining the crude. The crude product was purified by flash column chromatography on silica gel (toluene/ethyl acetate 95:5 to eliminate the impurities and then dichloromethane/methanol 90:10 to collect the product) obtaining 3.97 g of a yellow-orange oil (yield 97%).

Being Example 15 a mixture of different products, the ¹H-NMR was calculated giving to the signal at 7.86-8.03 ppm the value of 2.

¹H-NMR (DMSO-d₆, S ppm): 3.20-3.82 (m, 13.4H), 4.30-4.60 (m, 2H), 7.40-7.83 (m, 11H), 7.86-8.03 (m, 2H).

### Example 16

### Reaction Product of

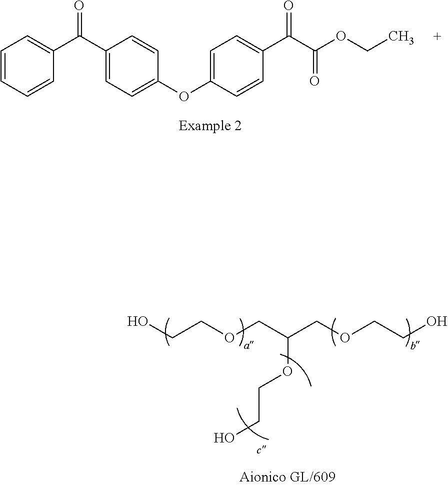

0.391 g (0.802 mmoles) of zirconium(IV) acetylacetonate were added under stirring to a warm solution of 3.00 g (8.013 mmoles) of Example 2 and 1.47 g (hydroxyl number 370 mg KOH/g)) of Aionico GL/609 (purchased from Lamberti SpA) in 30 mL of toluene. Then the reaction mixture was stirred at 105-110° C. for 2 hours eliminating ethanol by distillation. Progress of the reaction was monitored by TLC (toluene/ethyl acetate 95:5). Then additional 0.128 g of Aionico GL/609 and 0.196 g (0.402 mmoles) of zirconium(IV) acetylacetonate were added and the reaction mixture was stirred at 105-110° C. for further 3 hours eliminating ethanol by distillation. Then additional 0.196 g (0.402 mmoles) of zirconium(IV) acetylacetonate were added and the reaction mixture was stirred at 105-110° C. for further 3 hours eliminating ethanol by distillation. After completion of the reaction, the mixture was allowed to cool, diluted with ethyl acetate and washed in sequence with 50 mL (×2) of 1.5M hydrochloric acid, water and brine, respectively, dried over anhydrous sodium sulfate, filtered and the solvent removed by distillation under vacuum obtaining the crude. The crude product was purified by flash column chromatography on silica gel (toluene/ethyl acetate 95:5 to eliminate the impurities and then dichloromethane/methanol 90:10 to collect the product) obtaining 4.09 g of a yellow-orange oil (yield 99%).

Being Example 16 a mixture of different products, the ¹H-NMR was calculated giving to the signal at 7.96-8.12 ppm the value of 2.

¹H-NMR (DMSO-d₆, δ ppm): 3.20-3.82 (m, 13.4H), 4.30-4.60 (m, 2H), 7.20-7.34 (m, 4H), 7.46-7.61 (m, 2H), 7.62-7.78 (m, 3H), 7.78-7.89 (m, 2H), 7.96-8.12 (m, 2H).

### Example 17

### Reaction Product of

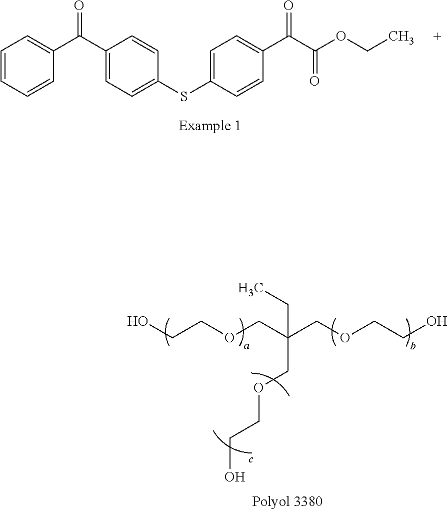

1.25 g (2.563 mmoles) of zirconium(IV) acetylacetonate were added under stirring to a warm solution of 10.00 g (25.611 mmoles) of Example 1 and 4.95 g (hydroxyl number 364 mg KOH/g) of Polyol 3380 (purchased from Perstorp) in 30 mL of toluene. Then the reaction mixture was stirred at 105-110° C. for 2.5 hours eliminating ethanol by distillation. Progress of the reaction was monitored by TLC (toluene/ethyl acetate 95:5). Then additional 0.625 g (1.282 mmoles) of zirconium(IV) acetylacetonate were added and the reaction mixture was stirred at 105-110° C. for further 1.5 hours eliminating ethanol by distillation. Then additional 5 mL of toluene and 0.625 g (1.282 mmoles) of zirconium(IV) acetylacetonate were added and the reaction mixture was stirred at 105-110° C. for further 3.5 hours eliminating ethanol by distillation. After completion of the reaction, the mixture was allowed to cool, diluted with ethyl acetate and washed with 100 mL (×2) of 3M hydrochloric acid. During the washings it was observed the formation of Zirconium precipitates that were removed by filtrating the biphasic mixture through a pad of celite. Then the organic layer was separated, washed with water and brine, dried over anhydrous sodium sulfate, filtered and the solvent removed by distillation under vacuum obtaining the crude. The crude product was purified by flash column chromatography on silica gel (toluene/ethyl acetate 95:5 to eliminate traces of LFC4563 and then eluted with dichloromethane/methanol 90:10) obtaining 13.76 g of a yellow-orange oil (yield 100%).

Being Example 17 a mixture of different products, the ¹H-NMR was calculated giving to the signal at 0.5-0.91 ppm the value of 3.

¹H-NMR (DMSO-d₆, δ ppm): 0.50-0.91 (m, 3H), 1.12-1.44 (m, 2H), 3.00-3.80 (m, 33.92H), 4.30-4.62 (m, 4.08H), 7.40-7.81 (m, 22.44H), 7.82-8.05 (m, 4.08H).

### Example 18

### Reaction Product of

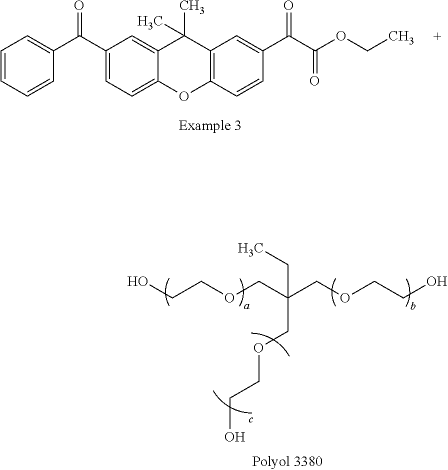

0.353 g (0.724 mmoles) of zirconium(IV) acetylacetonate were added under stirring to a warm solution of 3.00 g (7.238 mmoles) of Example 3 and 1.39 g (hydroxyl number 364 mg KOH/g) of Polyol 3380 (purchased from Perstorp) in 30 mL of toluene. Then the reaction mixture was stirred at 105-110° C. for 3.5 hours eliminating ethanol by distillation. Progress of the reaction was monitored by TLC (toluene/ethyl acetate 95:5). Then additional 0.177 g (0.363 mmoles) of zirconium(IV) acetylacetonate were added and the reaction mixture was stirred at 105-110° C. for further 1.5 hours eliminating ethanol by distillation. Then additional 0.177 g (0.363 mmoles) of zirconium(IV) acetylacetonate were added and the reaction mixture was stirred at 105-110° C. for further 1.5 hours eliminating ethanol by distillation. After completion of the reaction, the mixture was allowed to cool, poured into 50 mL of 2M hydrochloric acid and extracted with dichloromethane. The organic layer was separated, washed again with 50 mL of 2M hydrochloric acid, then with brine, dried over anhydrous sodium sulfate, filtered and the solvent removed by distillation under vacuum obtaining 4.10 g of a yellow-orange oil (yield 100%).

Being Example 18 a mixture of different products, the ¹H-NMR was calculated giving to the signal at 0.55-0.95 ppm the value of 3.

¹H-NMR (DMSO-d₆, δ ppm): 0.55-0.95 (m, 3H), 1.15-1.50 (m, 2H), 1.64 (m, 11.28H), 3.00-3.80 (m, 34.24H), 4.32-4.65 (m, 3.76H), 7.19-7.40 (m, 3.76H), 7.48-7.78 (m, 11.28H), 7.78-8.05 (m, 3.76H), 8.09-8.23 (m, 1.88H).

All compounds of Examples 1 to 14 may be reacted with a suitable polyol to provide a compound of the invention.

### Comparative Example

### Comparative Tests

The photoinitiators (PIs) of the invention were compared with Compound 8c of US2019/0155153 page 25 (COMP-1)

### Example 19

### Through-Cure and Surface Cure in Cyan Offset Ink Under LED 395 nm Lamp

The test formulations were prepared dissolving the photoinitiators at a concentration of 3% by weight (wt) in an industrial cyan offset ink. The coinitiator (Esacure A198) was added at the compositions in the same amount (3% by weight). The test formulations were homogenized with a mechanical stirrer for 1 hour at 50° C. and applied onto a white cardboard at 1.5 microns of thickness using IGT repro-tester equipment.

The formulations were cured using a LED 395 nm lamp (16 W/cm2) at a distance of 8 cm (Table 1).

The through cure test is a measurement of the complete ink cure obtained at a defined speed and checked by “thumb twist pressure test”. Higher speed corresponds to higher reactivity. The surface curing test is the measure of the surface curing at a fixed speed belt, it is measured in number of passages at 100 m/min. Lower is the number better is the surface curing.

The results are shown in Table 1:

TABLE 1

| | | Number of |
|---|---|---|
| | Through-cure | passages at |
| Photoinitiator | (m/min) | 100 m/min |
| EXAMPLE 17 | >100 | 5 |
| COMP-1 * | 77 | 7 |

* Comparative

This test confirms that compounds of formula (I) are more effective than the prior art photoinitiator.

### Example 20

### Yellow Index

The yellowing was measured as Yellow Index (YI) using a BYK color guide 45/0. The samples for the test were prepared as follows: a formulation containing the photoinitiators at 3% by wt, the coinitiator (Esacure A198) at 3% by wt, Photomer 6577 at 50% by wt, Photomer 4335 at 15% by wt, Photomer 4666 15% by wt, Photomer 4172 20% by wt was stirred for 1 hours at 60° C., spread with a thickness of 12 microns on a varnished cardboard using a bar-coater and cured with a Mercury Lamp (160 W/cm) at the maximum speed at which the tack-free is reached. Then, the yellowing as Yellow Index (YI) is measured, the results are shown in Table 2.

TABLE 2

| Photoinitiator | Yellow Index |
|---|---|
| EXAMPLE 15 | 7.99 |
| EXAMPLE 16 | 7.94 |
| EXAMPLE 17 | 6.36 |
| COMP-1 * | 9.62 |

* Comparative

All these tests confirm that compounds of formula (I) are more reactive than the compound of the prior art and surprising the good reactivity does not go with a high yellowing after curing.

1. A compound of formula (I) 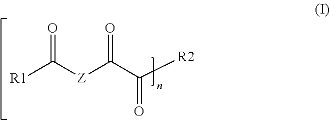
wherein
R1 is selected from aryl and heteroaryl, each being unsubstituted or substituted by one or more substituents selected from the group consisting of:
halogen, substituted or unsubstituted C1-C20 alkyl, substituted or unsubstituted C1-C20 alkoxy, substituted or unsubstituted C1-C20 alkylthio, substituted or unsubstituted (C1-C12alkyl)₂amino, morpholino, piperidino, piperazino and N(C1-C12alkyl)piperazino;
n is from 2 to 8;
R2 comprises C1-C50 alkyl which is interrupted by one or more oxygens; or a group of formula (II)
*-G-[OH]p (II)
wherein
G comprises residue of monomeric, oligomeric or polymeric polyols G-(OH)ₙ₊ₚ;
p is from 0 to 7;
n+p is from 2 to 8;
provided that when R2 is a C1-C50 alkyl which is interrupted by one or more oxygens, then n is 1;
the star of formula (II) indicates a linking bond to the compound of formula (I);
Z is selected from structure (i), (ii) and (iii) 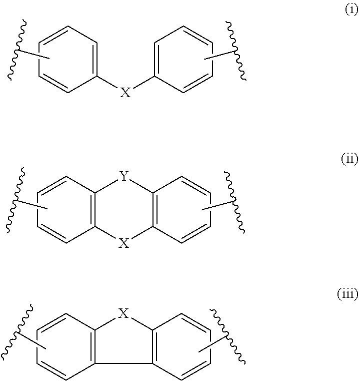
wherein
X comprises O, S or C(R3)(R4);
Y comprises O, S, C(R3)(R4) or NR5;
R3 and R4 each comprise H, C1-C12 alkyl, OH or C1-C10 alkoxy;
R5 comprises H or C1-C8 alkyl;
and the wavy line in each of structures (i), (ii) and (iii) indicates bonds linking to the keto groups of formula (I);
provided that when Z is structure (iii) and X is C(R4)(R5), then R1 is a heteroaryl comprising a substituted coumarin or a benzocoumarin.

2. The compound of claim 1, wherein said G-(OH)ₙ₊ₚ is selected from the group consisting of a monomeric polyol, an oligomeric polyol and mixtures thereof.

3. The compound of claim 1, wherein said G-(OH)ₘ₊ₙ₊ₚ has a number average molecular weight not greater than 1,500 Da.

4. The compound of claim 1, wherein
n+p is from 2 to 8;
n is from 2 to 6; and
p is from 0 to 6.

5. The compound of claim 1, wherein said Z comprises structure (i) and X comprises S or O.

6. The compound of claim 1, wherein said R1 is selected from the group consisting of phenyl, trimethylphenyl, halophenyl fluorophenyl, biphenyl, coumarin-3-yl, naphthyl and thiophenyl.

7. The compound of claim 1, wherein the compound comprises a reaction product of 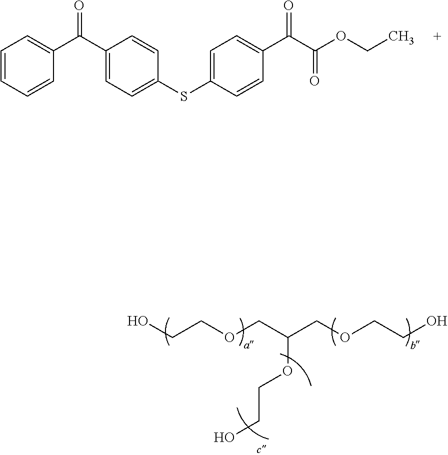
or a reaction product of 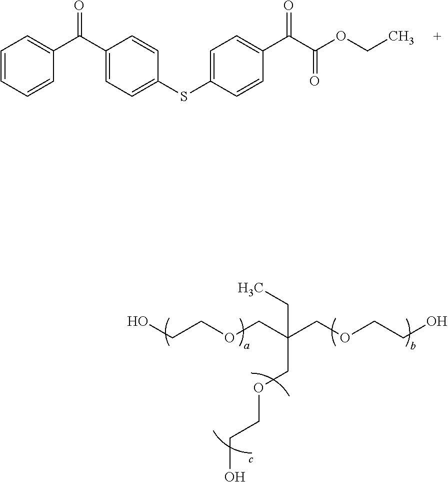
wherein (a), (b), (c) and (d); and (a″), (b″), (c″) and (d″) are each 0 to 15.

8. The compound of claim 1, from said compound comprising one of 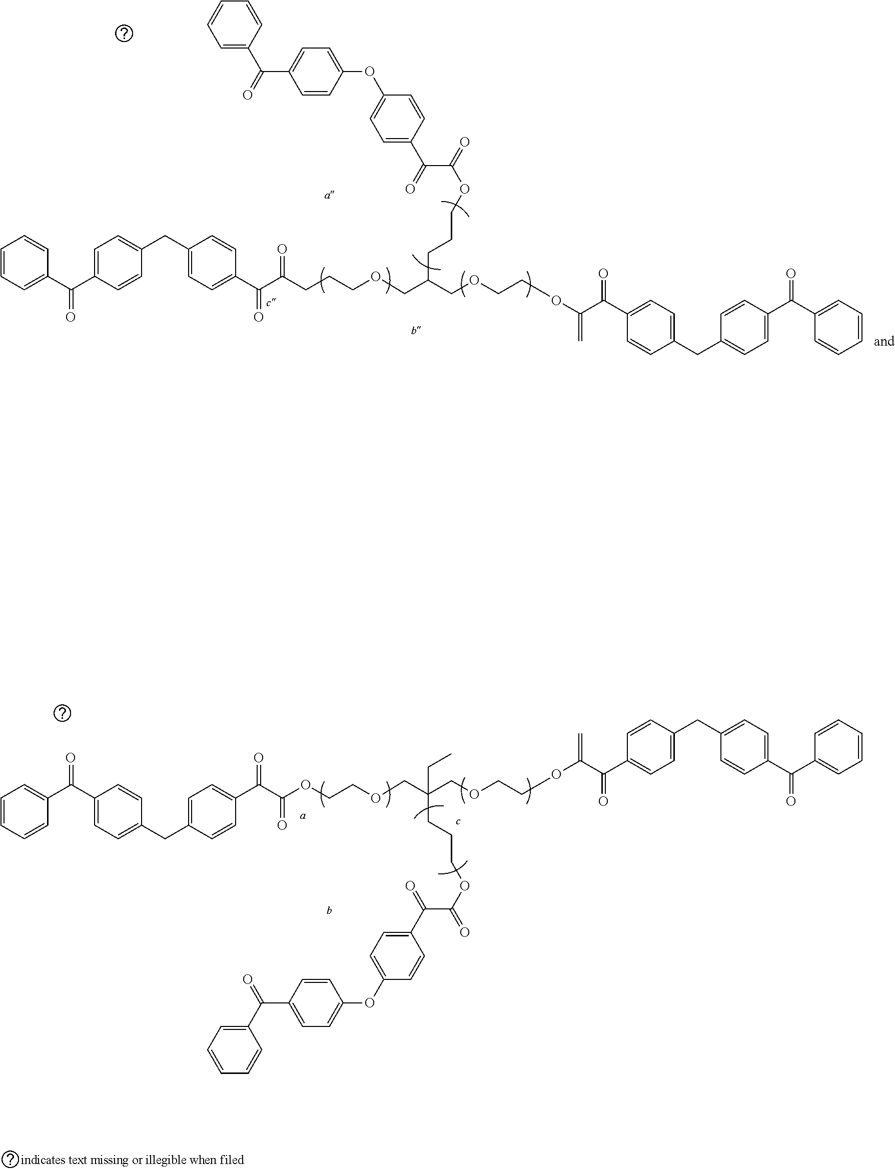
wherein (a), (b), (c) and (d); and (a″), (b″), (c″) and (d″) are, each independently, 0 to 15.

9. A photopolymerizable composition comprising:
a) from 50 to 99.9% by weight, based on the total content of the photopolymerizable composition of at least one ethylenically unsaturated compound;
b) from 0.1 to 35% by weight, based on the total content of the photopolymerizable composition, of at least one compound of formula (I), as defined in claim 1; and
c) from 0 to 20% by weight, based on the total content of the photopolymerizable composition, of an accelerator and/or of a coinitiator.

10. The photopolymerizable composition of claim 9, further comprising one or more of:
d) from 0.01 to 15% by weight based on the total content of the photopolymerizable composition, of one or more photosensitizer; and
e) from 0.5 to 15% by weight based on the total content of the photopolymerizable composition, one or more further photoinitiators.

11. The photopolymerizable composition of claim 9, wherein said accelerator and/or coinitiator is an amine.

12. A process for photocuring photopolymerizable compositions, coatings, adhesives and inks, said process comprising:
(i) providing a photopolymerizable composition as defined in claim 9;
(ii) coating or printing said photopolymerizable composition onto a substrate; and
(iii) photopolymerizing said coated or printed photopolymerizable composition on said substrate with a light source.

13. (canceled)

14. The process of claim 12, wherein said light source comprises UV light in at least one of the UVA, UVB and UVC ranges.

15. The process of claim 12 wherein said light source is an LED source emitting in the range of 350 to 420 nm.

16. (canceled)

17. (canceled)

18. The compound of claim 1, wherein said G-(OH)ₙ₊ₚ is ethoxylated or propoxylated and comprises a monomeric polyol, an oligomeric polyol or a mixture thereof.

19. The compound of claim 1, wherein
n+p is from 3 to 6;
n is from 2 to 4; and
p is from 0 to 3.

20. The compound of claim 1 wherein said G-(OH)ₘ₊ₙ₊ₚ has a number average molecular weight not greater than 1,000 Da and not lower than 200 Da.

21. The photopolymerizable composition of claim 9, wherein said accelerator and/or coinitiator is a tertiary amine.

22. The compound of claim 7, wherein (a′), (b′), (c′) and (d′) are, each independently, 1 to 8.

23. The photopolymerizable composition of claim 9 comprising:
a) from 70 to 98.9% by weight, based on the total content of the photopolymerizable composition of at least one ethylenically unsaturated compound;
b) from 0.2 to 15% by weight, based on the total content of the photopolymerizable composition, of at least one compound of formula (I), as defined in claim 1; and
c) from 0.2 to 15% by weight, based on the total content of the photopolymerizable composition, of an accelerator and/or of a coinitiator.

24. The compound of claim 1, wherein said polyol G-(OH)ₘ₊ₙ₊ₚ is ethoxylated or propoxylated.
